SEQUENCE LISTING (1 sequence)

SEQ ID NO: 1 - protein, 192 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

(19) United States
(12) Patent Application Publication
(10) Pub. No.: US 2015/0335671 A1
(43) Pub. Date: Nov. 26, 2015

(54) SMALL-MOLECULE BINDING SITE ON PRO-APOPTOTIC BAX REGULATES INHIBITION OF BAX ACTIVITY

(71) Applicant: ALBERT EINSTEIN COLLEGE OF MEDICINE OF YESHIVA UNIVERSITY, Bronx, NY (US)

(72) Inventors: Evripidis Gavathiotis, New York, NY (US); Thomas Garner, Bronx, NY (US)

(73) Assignee: ALBERT EINSTEIN COLLEGE OF MEDICINE OF YESHIVA UNIVERSITY, Bronx, NY (US)

(21) Appl. No.: 14/760,741

(22) PCT Filed: Jan. 13, 2014
(86) PCT No.: PCT/US2014/011213

§ 371 Date: Jul. 14, 2015

Related U.S. Application Data
(60) Provisional application No. 61/752,104, filed on Jan. 14, 2013.

(51) Int. Cl. A61K 31/7028; A61K 31/496; A61K 31/325; A61K 31/47; A61K 31/4985; G01N 33/50; A61K 31/506; A61K 31/53; A61K 31/546; A61K 31/5377; A61K 31/675; A61K 31/11; A61K 31/4709

(52) U.S. Cl. CPC A61K 31/7028; A61K 31/11; A61K 31/496; A61K 31/325; A61K 31/47; A61K 31/4985; A61K 31/4709; A61K 31/506; A61K 31/53; A61K 31/546; A61K 31/5377; A61K 31/675; G01N 33/5008; G01N 2510/00

(57) ABSTRACT

Methods are provided for identifying an agent as a selective inhibitor of a Bcl-2-associated x-protein (BAX).

### CROSS-REFERENCE TO RELATED APPLICATIONS

This application claims benefit of U.S. Provisional Application No. 61/752,104, filed Jan. 14, 2013, the contents of which are hereby incorporated by reference.

### STATEMENT OF GOVERNMENT SUPPORT

This invention was made with government support under grant number R00 HL095929 awarded by the National Institutes of Health. The government has certain rights in this invention.

### BACKGROUND OF THE INVENTION

Throughout this application various publications are referred to by number in parentheses. Full citations for these references may be found at the end of the specification. The disclosures of these publications, and all patents, patent application publications and books referred to herein, are hereby incorporated by reference in their entirety into the subject application to more fully describe the art to which the subject invention pertains.

Apoptosis is an evolutionarily conserved process that plays a critical role in embryonic development and tissue homeostasis. The dysregulation of apoptosis is pivotal to a number of high mortality human pathogenesis including cancer, cardiovascular diseases, and neurodegenerative diseases. The BCL-2 family includes both pro- and anti-apoptotic proteins that form a complex protein interaction network of checks and balances that dictate cell fate. The pro-apoptotic BCL-2 proteins, Bcl-2-associated x-protein (BAX) and BAK, induce mitochondrial outer-membrane permeabilization and represent the key gatekeepers and effectors of mitochondrial apoptosis. Thus, inhibition of pro-apoptotic BAX or BAK impairs the cells' ability to initiate premature or unwanted cell death in terminally differentiated cells, including cardiomyocytes and neurons.

This laboratory has previously identified a number of key structural regions of BAX involved in the regulation of BAX activation, including the BAX trigger site. However, the molecular basis for, and site of, inhibition of BAX is unknown.

The present invention discloses assays for identifying selective and competitive BAX inhibitors.

### SUMMARY OF THE INVENTION

A method is provided for identifying an agent as an inhibitor of a Bcl-2-associated x-protein (BAX) comprising

a) contacting the BAX with the agent and determining if the agent inhibits truncated BH3 interacting-domain death agonist (tBID)-induced BAX activation and/or BAX-mediated pore formation, and
b) identifying, for an agent identified in step a) as inhibiting tBID-induced BAX activation and/or BAX-mediated pore formation, the site on the BAX to which the agent binds, and determining if said site substantially overlaps, or is co-extensive with, a binding site comprised by helices α3, α4, α5 and loop α3-α4 of BAX,
wherein an agent that both (i) inhibits tBID-induced BAX activation and/or BAX-mediated pore formation and (ii) binds to a site that substantially overlaps, or is co-extensive with, a binding site comprised by helices α3, α4, α5 and loop α3-α4 of BAX, is identified as an inhibitor of BAX.

A method is also provided of identifying an agent as an inhibitor of a Bcl-2-associated x-protein (BAX) comprising contacting the BAX with the agent and iMAC1 or iMAC2 (see FIG. 2 for structures) and experimentally determining if the agent inhibits the BAX competitively in relation to iMAC1 or iMAC2, wherein an agent that competitively inhibits BAX in relation to iMAC1 or iMAC2 is identified as an inhibitor of BAX.

A method is also provided of identifying an agent as a selective inhibitor of a Bcl-2-associated x-protein (BAX) comprising determining in silico if a molecular model of the agent will bind to a site of binding on the BAX that substantially overlaps or is co-extensive with a binding site comprised by helices α3, α4, α5 and loop α3-α4 of BAX, and when such an agent is determined to bind to the site, identifying the agent as a selective inhibitor of a BAX.

A method is also provided of identifying an agent as a selective inhibitor of a BCL-2, BCL-XL, MCL-1, BCL-B, BCL-w, or BFL-1/A1 comprising

a) contacting the BCL-2, BCL-XL, MCL-1, BCL-B, BCL-w, or BFL-1/A1 with the agent and determining if the agent inhibits BCL-2, BCL-XL, MCL-1, BCL-B, BCL-w, or BFL-1/A1 activity, and
b) identifying the site of binding on a BCL-2, BCL-XL, MCL-1, BCL-B, BCL-w, or BFL-1/A1 of an agent identified in step a) as inhibiting BCL-2, BCL-XL, MCL-1, BCL-B, BCL-w, or BFL-1/A1, and determining if the site of binding substantially overlaps, or is co-extensive with, a binding site structurally equivalent to a binding site on a BAX comprised by helices α3, α4, α5 and loop α3-α4 of BAX,
wherein an agent that both (i) inhibits BCL-2, BCL-XL, MCL-1, BCL-B, BCL-w, or BFL-1/A1 and (ii) binds to a site structurally equivalent to a binding site on a BAX comprised by helices α3, α4, α5 and loop α3-α4 of BAX, is identified as a selective inhibitor of BCL-2, BCL-XL, MCL-1, BCL-B, BCL-w, or BFL-1/A1, respectively.

Also provided is a method of inhibiting Bcl-2-associated x-protein (BAX) comprising contacting the BAX with a compound having a structure listed in Table 1, or a pharmaceutically acceptable salt thereof or stereoisomer thereof, in an amount effective to inhibit BAX:

Also provided is a method of treating hypoxic cardiomyocytes or cardiac ischemia-reperfusion injury in a subject comprising administering to the subject an amount of a compound having a structure listed in Table 1, or an amount of iMAC1 or an amount of iMAC2, or a pharmaceutically acceptable salt of any thereof or stereoisomer thereof, in an amount effective to treat hypoxic cardiomyocytes or cardiac ischemia-reperfusion injury, respectively.

Also provided is a method of treating a condition associated with excessive BAX in a subject comprising administering to the subject an amount of a compound having a structure listed in Table 1, or an amount of iMAC1 or an amount of iMAC2, or a pharmaceutically acceptable salt of any thereof or stereoisomer thereof, in an amount effective to treat a condition associated with excessive BAX.

Also provided is a method of identifying a compound as an inhibitor of a Bcl-2-associated x-protein (BAX) comprising

a) in silico modelling a pharmacophore for a binding site on BAX comprised by helices α3, α4, α5 and loop α3-α4 of BAX,
b) conducting an in silico comparison of the 3D structure of the compound with the modeled pharmacophore;
c) identifying the compound as an inhibitor or not of BAX,
wherein a compound having a 3D structure that matches or substantially overlaps the modeled pharmacophore is identified as an inhibitor of BAX, and a compound having a 3D structure that does not match or does not overlap the modeled pharmacophore is not identified as an inhibitor of BAX.

### BRIEF DESCRIPTION OF THE DRAWINGS

FIG. 1. The BAX trigger site (blue) is a recently discovered binding site for BH3-only proteins such as BIM and tBID, which results in activation of BAX, followed by its translocation to the outer mitochondrial membrane and release of apoptogenic factors. In contrast, the canonical BH3-binding pocket, identified in anti-apoptotic proteins, formed by BH domains 1-3 (green), maps to the opposite side of BAX, but remains occupied by the C-terminal helix 9 (yellow) when the protein is in the inactive, monomeric state (4). Finally the BH3 domain (dark green) of BAX is known to form an inhibitory complex with anti-apoptotic Bcl-2 family proteins, this interaction site is buried in inactive monomeric BAX and is only exposed after activation.

FIG. 2. Chemical structures of iMACs (inhibitors of mitochondrial apoptosis-induced channel) used in the studies. iMAC1, iMAC2 and iMAC3 are more potent inhibitors of cytochrome c release and cell-based assays for mitochondrial apoptosis-induced channel activity (Peixoto et al. Biochem J. 2009)

FIG. 3. Left: Kinetic trace of percent release of ANTS and DPX from liposomes in response to tBID activated BAX in the absence (blue) and presence of iMAC1 (green) and iMAC2 (Red). Right: Percent release after 20 minutes at different doses of iMAC1 (green), iMAC2 (red) and iMAC3 (blue). Each molecule was tested at 6, 3 and 1.5 μM, high to low doses are indicated by dark to light tones. Liposomal release assay demonstrates the capacity of iMAC1 and iMAC2, but not iMAC3, to inhibit tBID-induced BAX activation and BAX pore formation and block release of fluorescent molecules encapsulated in liposomal membranes.

FIG. 4A-B. Measured chemical shift changes of 15N-BAX upon iMAC1 and iMAC2 titration up to a ratio of 1:4 BAX:iMAC are plotted as a function of BAX residue number. Residues with significant backbone amide chemical shift change are highlighted in orange. Highest significant chemical shift changes (twice the calculated significance threshold) are highlighted in red. Chemical shift changes are concentrated in the region formed by helices α3, α4, α5 and loop α3-α4. Chemical shift changes in other regions of the structure are not localized and predominantly occur in hydrophobic residues at the hydrophobic core of the BAX structure. Regions that previously identified to be involved in regulating BAX activation are shown in violet shading (helix α1 and α6), teal shading (helix α2) and yellow shading (helix α9).

FIG. 5 Significant CSP mapped onto the surface and ribbon depictions of BAX, the BAX trigger site (blue), the canonical BH3-binding site and α9 (green) are included for comparison. Affected residues are represented by the orange and red spheres in the ribbon diagram and by the orange and red bars in the plot (the calculated significance threshold is marked in orange and is equal to the average shift plus the standard deviation while values at double the threshold are marked in red).

FIG. 6A-6B. Cartoon representation of the BAX structure showing residues that undergo significant chemical shift changes (orange) and highest significant chemical shift changes (red) upon iMAC1 and iMAC2 binding. Select helices regulating BAX activation are colored in violet (helix α1 and α6), teal (helix α2) and yellow (helix α9).

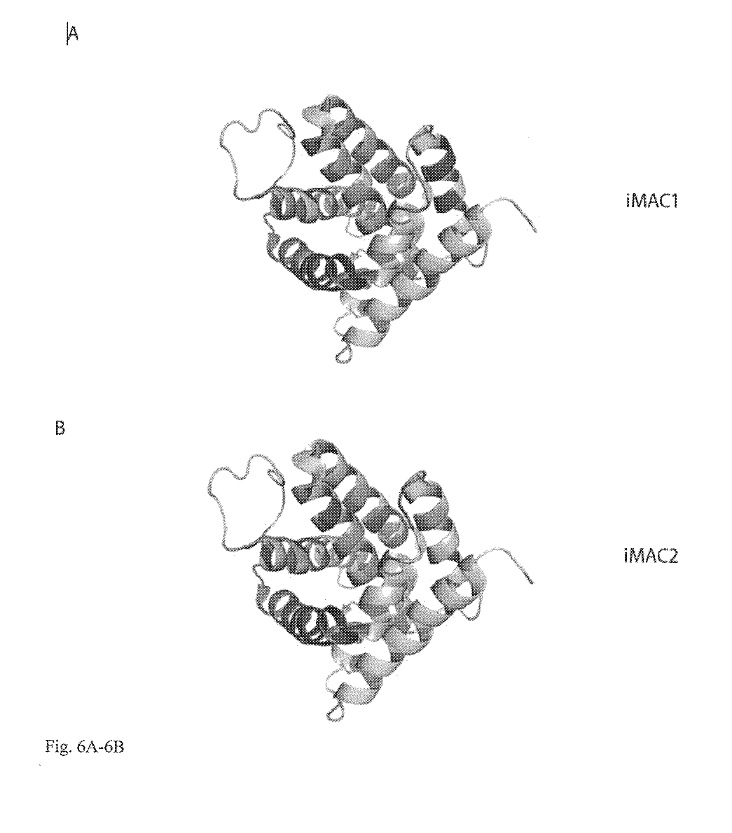

FIG. 7A-7B. Surface representation of the BAX structure with mapped residues in orange (significant chemical shift changes) and highest significant chemical shift changes (red) concentrated in helices α3, α4, α5 and loop α3-α4 are shown for the titrations of iMAC1 and iMAC2. The most energetically favorable docked structures of iMAC1 and iMAC2 calculated by the molecular docking software Glide are shown to overlap with BAX interacting residues identified by the NMR studies.

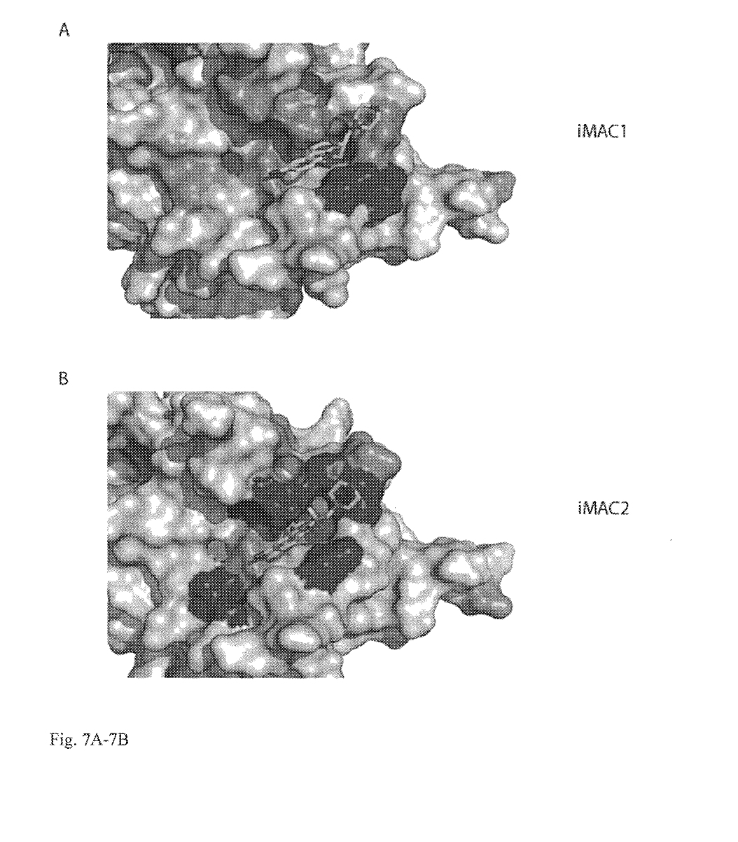

FIG. 8A-8B. NMR CSP-guided in silico docking of iMAC1 (green) and iMAC2 (red) with the BAX monomer NMR structure (PDB ID: 1F16) using Glide (Maestro, Schrodinger). (A) The lowest energy docking pose is consistent with the observed NMR CSP data (orange and red). (B) Key proposed interaction with the iMAC molecules, a number of hydrophobic contacts, cation-pi and electro static interactions contribute to the proposed binding site.

FIG. 9A-9C. Structures of BAX with most favorable docked poses of iMAC1 and iMAC2 showing the location and interacting residues of the novel BAX binding site comprised by helices α3, α4, α5 and loop α3-α4.

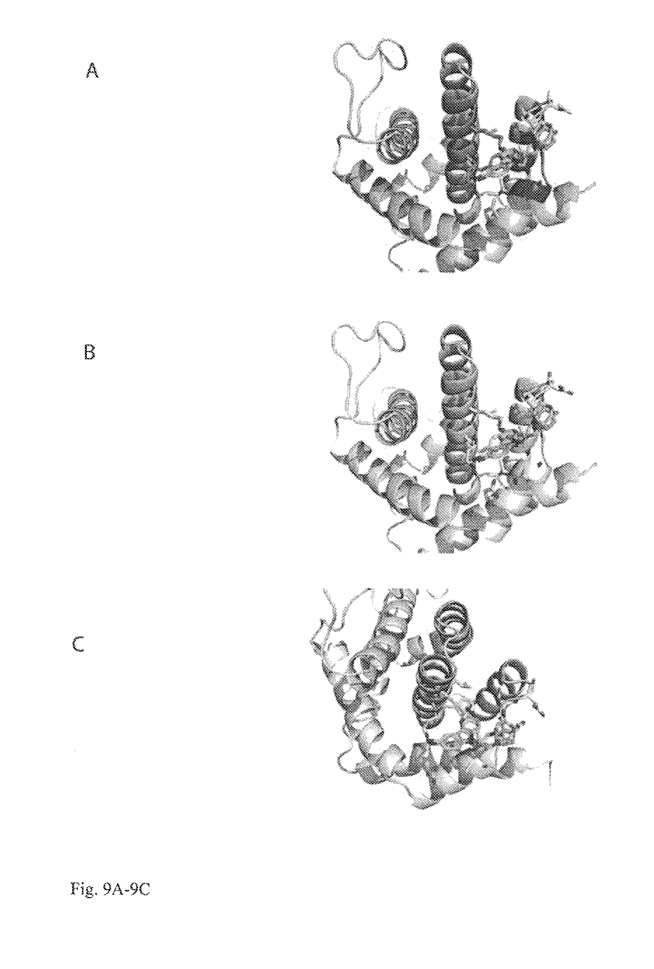

FIG. 10. Close view of the novel BAX binding site and bound docked structures of iMAC1 (orange) and iMAC2 (cyan). Compounds form favorable hydrophobic contacts with the side chain of hydrophobic residues 180, L120, F116, and A81, a strong I-cation interaction between one phenyl ring of the iMACs and the side-chain of K123, and a salt-bridge between the amino group of the iMACs' piperazine ring and the carboxyl group of D86.

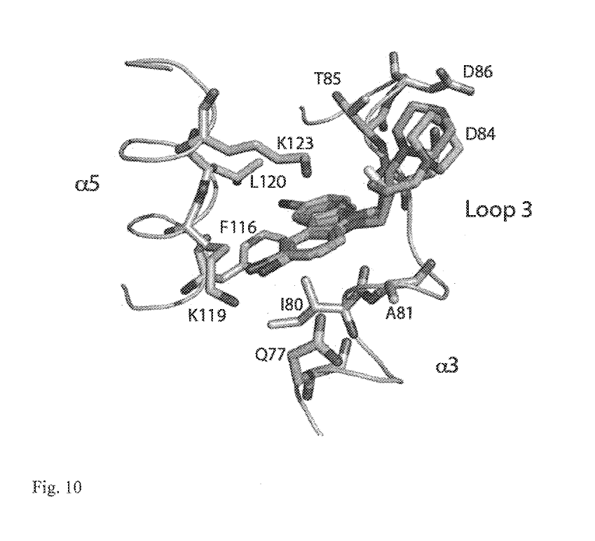

FIG. 11. Cartoon and surface representation of the proposed novel BAX binding site indicated by NMR and docking studies. Residues highlighted in red undergo chemical shift changes and interact with the docked structures of iMACs. Residues highlighted in yellow are proposed to be part of the binding site but do not directly interact with iMACs. The binding site is comprised by residues Q76, 180, A81, V83, D84, T85, D86, P88, V91, F92, V95, F116, L119, L120 and K123.

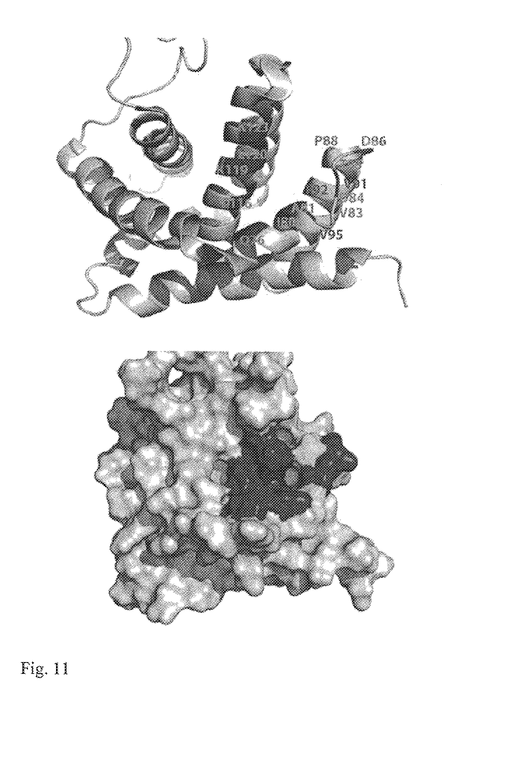

FIG. 12: Chemical shift changes of the backbone amide for residue A81 (highest chemical shift change) monitored by titration of iMAC1, iMAC2 and iMAC3 to the apo of BAX. iMAC1 and iMAC2 demonstrate similar binding potency as evidenced by the degree of chemical shift change over 4:1 iMAC:BAX ratio, whereas iMAC3 shows no binding to the proposed BAX binding site.

FIG. 13: Measured chemical shift changes of ¹⁵N-BAX upon octyl glucoside detergent titration, below the detergent's CMC point, are plotted as a function of BAX residue number. Residues with significant backbone amide chemical shift change are highlighted in orange. Highest significant chemical shift changes (twice the calculated significance threshold) are highlighted in red. Chemical shift changes are concentrated in the region formed by helices α3, α4, α5, α6 and loop α3-α4 showing a significant overlap with residues involved in binding interaction with iMAC1 and iMAC2.

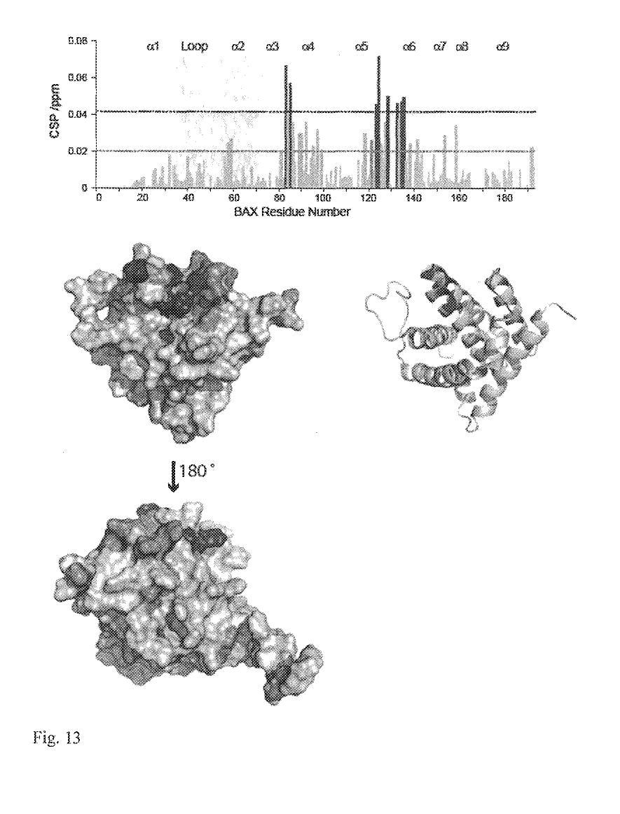

FIG. 14A-14B. 20 ns molecular dynamics (MD) simulations were performed on the BAX-iMAC1 complex using Desmond (Maestro, Schrodinger) at 300 K and 350 K. After 20 ns of MD simulation, iMAC1 remains bound to the proposed binding pocket in contact with the same residues (A). Comparison of the MD derived B-factors from 20 ns simulations of BAX with and without iMAC1 show evidence for reduced motions in several important regions of secondary structure, including the BH3 domain and helix 9, in the presence of iMAC1 (B).

FIG. 15. Further binding site residues highlighted in red color. Residues further include R34, D71, S72, M74, Q77, R78, 180, A81, A82, V83, D84, T85, D86, S87, P88, R89, V91, F92, V95, Y115, F116, S118, K119, L120, L122, K123, A124, C126, T127, K128, V129, L132, T135, 1136, W139.

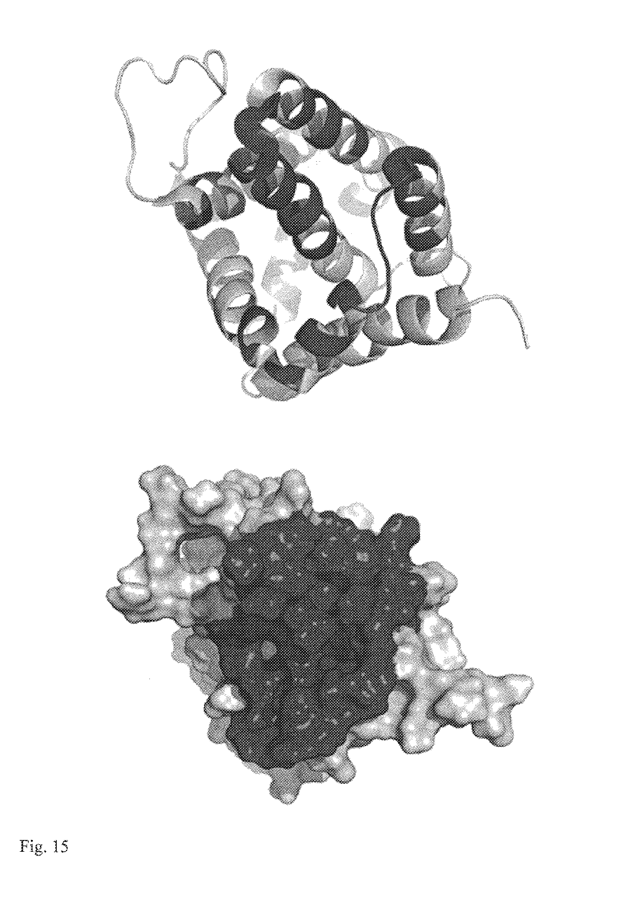

FIG. 16. Discovery of small molecules that bind to the novel BAX site through in silico screening: BAX structure (grey) in cartoon representation (top) and surface representation (bottom) highlighting the novel BAX inhibitory site (blue) and a group of small molecules from the screen, engaging the site in a variety of different interactions with BAX residues.

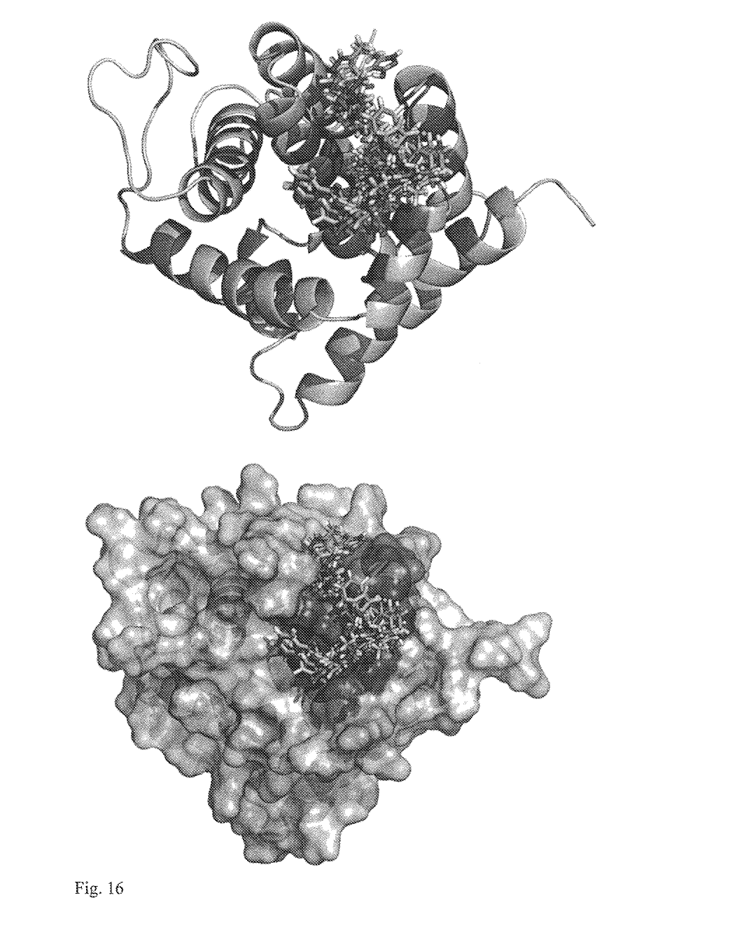

FIG. 17. Same as FIG. 16 but the docked structures of small molecules on BAX are shown from a different viewing angle.

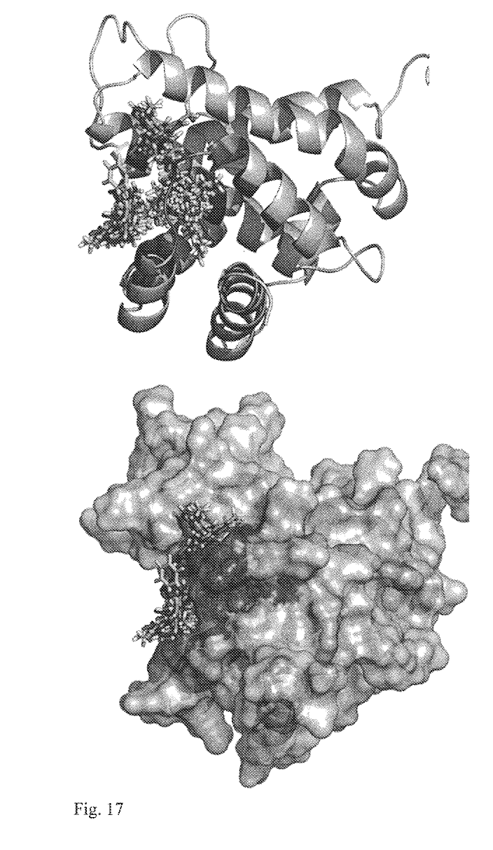

FIG. 18A-18B. A) n-octyl-3-D-glucoside inhibits BAX activation and BAX-mediated pore formation in liposomal membranes at concentrations well below the critical micelle concentration (CMC). B) Docked structure of n-octyl-3-D-glucoside within the novel BAX site. FIG. 13 also shows the NMR analysis of n-octyl-o-D-glucoside binding to BAX that demonstrates binding to the novel site that regulates BAX inhibition.

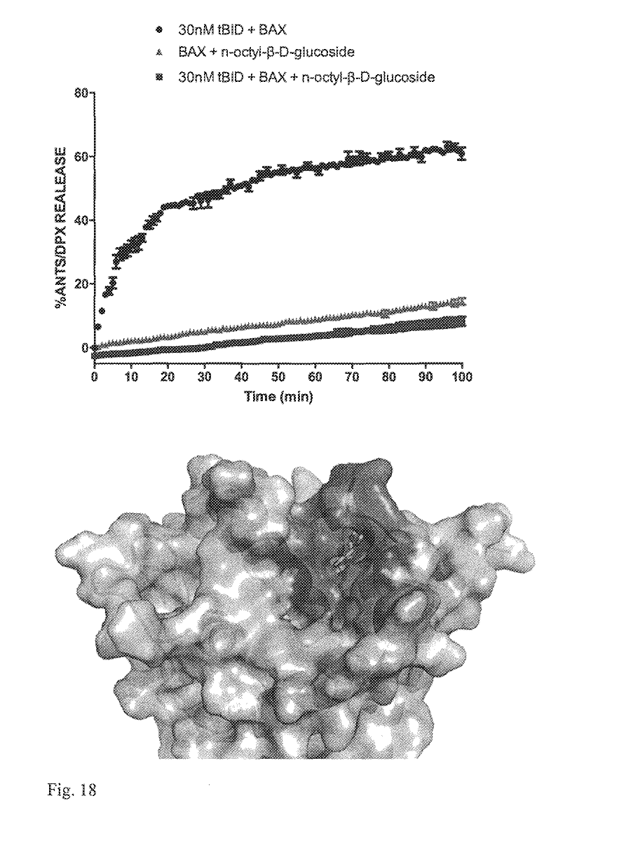

FIG. 19. iMAC1 inhibits BAX translocation to liposomal membranes induced by tBID activation. Liposomes containing the fluorophore, ANTS, and the quencher DPX, were incubated with 800 nM BAX. Addition of 24 nM tBID activated BAX resulting in translocation of soluble BAX to the liposomal membrane. Liposomes were separated from soluble BAX using a 3 ml Sepharose CL-2B column and analyzed by western blot. iMAC1 inhibits tBID-induced Bax translocation into liposomes.

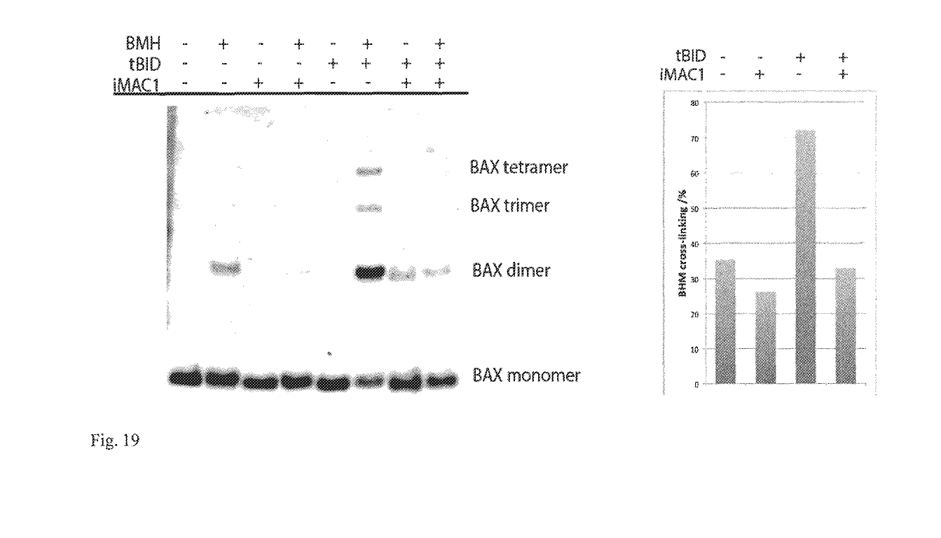

FIG. 20. iMAC1 inhibits BAX oligomerization to liposomal membrane induced by tBID activation. Liposomes were incubated with 0.2-1 μM BAX. Addition of 30 nM tBID activated BAX resulting in BAX-mediated pore formation within the liposomal membrane. The compound Bismaleimidohexane (BMH) was used to irreversibly cross-link oligomeric BAX resulting in the observation of dimeric and multimeric BAX in denaturing SDS PAGE gels. iMAC1 inhibits the formation of tBID induced BAX oligomers in liposomes.

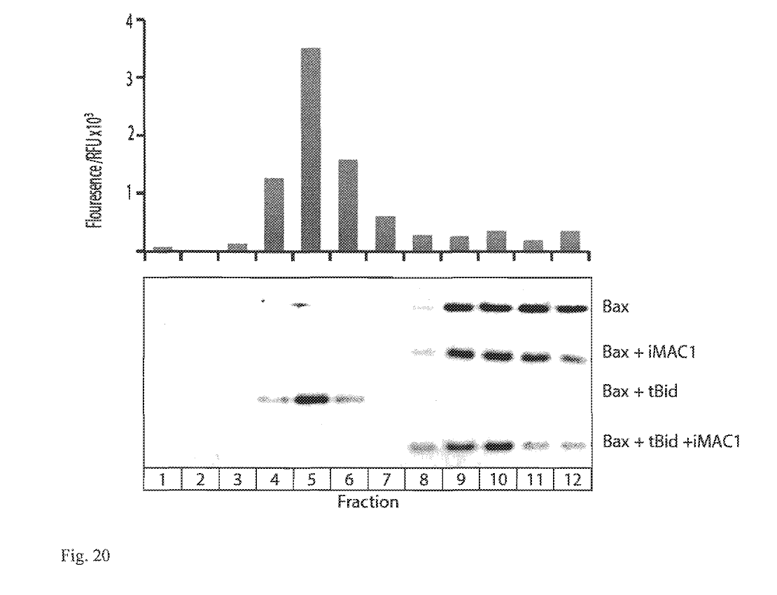

FIG. 21. iMAC1 protects cardiac myocytes from cell death induced by hypoxia as compared to vehicle (0.2% DMSO), whereas iMAC1 has no effect on cardiac myocytes under normoxia conditions.

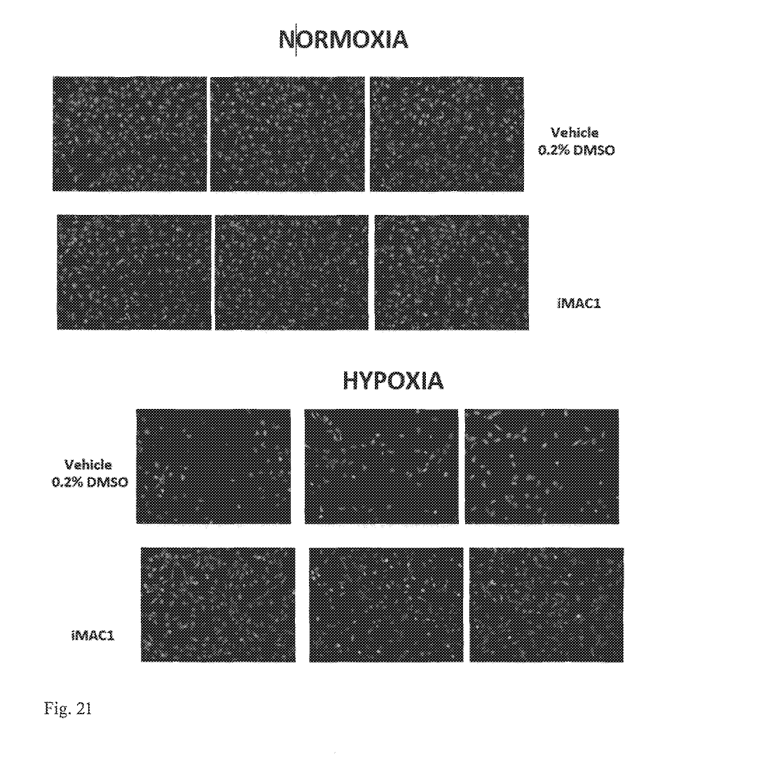

FIG. 22. iMACs bind to both monomer and oligomeric BAX. Ligand-observed NMR of the aromatic peaks of iMAC2 show chemical shift changes, protein to ligand STD transfer, and WATER LOGSY transfers demonstrating binding to both oligomeric BAX (A) and monomeric BAX (B).

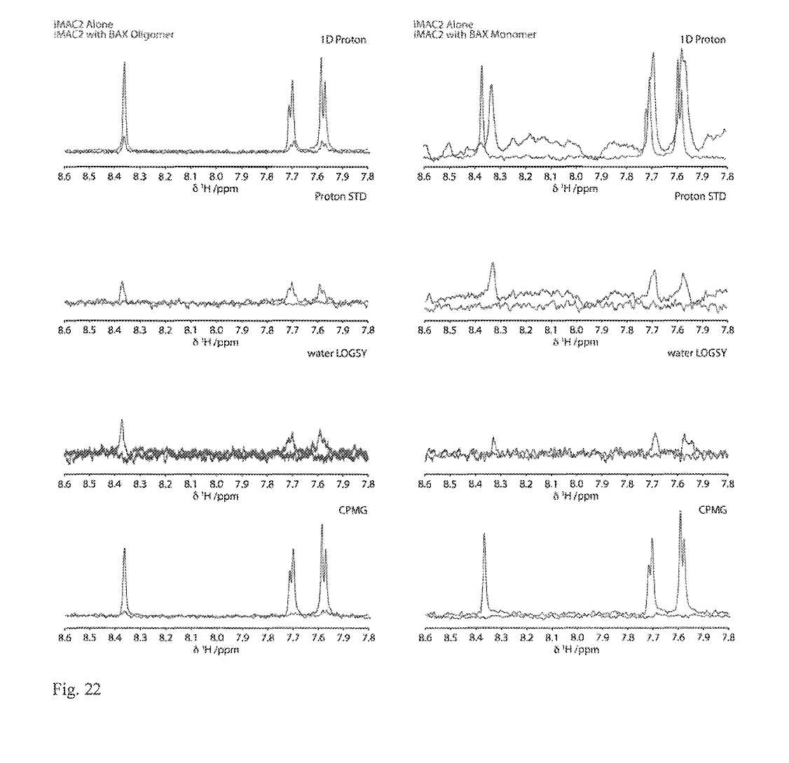

### DETAILED DESCRIPTION OF THE INVENTION

A method is provided for identifying an agent as an inhibitor of a Bcl-2-associated x-protein (BAX) comprising

a) contacting the BAX with the agent and determining if the agent inhibits truncated BH3 interacting-domain death agonist (tBID)-induced BAX activation and/or BAX-mediated pore formation, and
b) identifying, for an agent identified in step a) as inhibiting tBID-induced BAX activation and/or BAX-mediated pore formation, the site on the BAX to which the agent binds, and determining if said site substantially overlaps, or is co-extensive with, a binding site comprised by helices α3, α4, α5 and loop α3-α4 of BAX,
wherein an agent that both (i) inhibits tBID-induced BAX activation and/or BAX-mediated pore formation and (ii) binds to a site that substantially overlaps, or is co-extensive with, a binding site comprised by helices α3, α4, α5 and loop α3-α4 of BAX, is identified as an inhibitor of BAX.

In an embodiment, the BAX binding site comprised by helices α3, α4, α5 and loop α3-α4 of BAX comprises one or more of residues Q76, 180, A81, V83, D84, T85, D86, P88, V91, F92, V95, F116, L119, L120 and K123. In an embodiment, the BAX binding site comprises all of residues Q76, 180, A81, V83, D84, T85, D86, P88, V91, F92, V95, F116, L119, L120 and K123. In an embodiment, the BAX binding site comprised by helices α3, α4, α5 and loop α3-α4 of BAX also comprises one or more of residues R34, D71, S72, M74, Q77, R78, 180, A81, A82, V83, D84, T85, D86, S87, P88, R89, V91, F92, V95, Y115, F116, S118, K119, L120, L122, K123, A124, C126, T127, K128, V129, L132, T135, 1136, and W139.

In an embodiment, the identifying and/or determining step(s) are experimentally or empirically performed.

In an embodiment, the identifying for an agent the site on the BAX to which the agent binds, and determining if said site substantially overlaps, or is co-extensive with, a binding site comprised by helices α3, α4, α5 and loop α3-α4 of BAX is effected using NMR chemical shift perturbations (“NMR CSP”). In an embodiment, the identifying for an agent the site on the BAX to which the agent binds, and determining if said site substantially overlaps, or is co-extensive with, a binding site comprised by helices α3, α4, α5 and loop α3-α4 of BAX is effected using paramagnetic NMR. In an embodiment, the identifying for an agent the site on the BAX to which the agent binds, and determining if said site substantially overlaps, or is co-extensive with, a binding site comprised by helices α3, α4, α5 and loop α3-α4 of BAX is effected using crystallography.

In an embodiment, the BAX is monomeric BAX. In an embodiment, the BAX is inactive when contacted with the agent. In an embodiment, the BAX is a human BAX. In an embodiment, the BAX is an alpha isoform BAX.

In an embodiment, the inhibition of tBID-induced BAX activation and/or BAX pore formation is determined by a membrane-bound release assay. In an embodiment, the inhibition of tBID-induced BAX activation and/or BAX pore formation is determined by a liposonal-release assay. The liposome can comprise the BAX. The release can be of a traceable marker, such as, in non-limiting embodiments, a dye or a fluorophore. tBID administered so as to activate the BAX. BAX activation and pore-formation can be determined directly or indirectly by methods known in the art. In an embodiment, inhibition of tBID-induced BAX activation and/or BAX-mediated pore formation is determined by quantifying the reduction in release of fluorescent molecules encapsulated in liposomal membranes comprising BAX.

In an embodiment, identifying the site of binding on the BAX comprises determining chemical shift changes of 15N-BAX upon a detergent titration, below the detergent's critical micellular concentration (CMC) point, at one or more residues of the BAX. In an embodiment, the detergent comprises octyl glucoside.

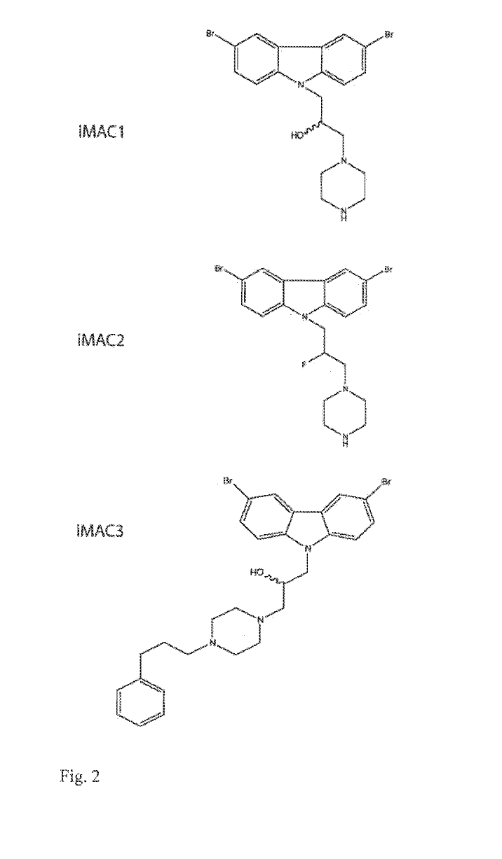

A method is also provided of identifying an agent as an inhibitor of a Bcl-2-associated x-protein (BAX) comprising contacting the BAX with the agent and iMAC1 or iMAC2 (see FIG. 2 for structures) and experimentally determining if the agent inhibits the BAX competitively in relation to iMAC1 or iMAC2, wherein an agent that competitively inhibits BAX in relation to iMAC1 or iMAC2 is identified as an inhibitor of BAX.

In an embodiment, an agent that does not competitively inhibit BAX in relation to iMAC1 or iMAC2 is not identified as a selective inhibitor of BAX. Competitive inhibition is measured by techniques known in the art, including comprising varying concentrations of the agent and the iMAC1 or iMAC2 and determining relevant parameters, such as one or more of Kᵢ, ½ Vmax.

A method is also provided of identifying an agent as a selective inhibitor of a Bcl-2-associated x-protein (BAX) comprising determining in silico if a molecular model of the agent will bind to a site of binding on the BAX that substantially overlaps or is co-extensive with a binding site comprised by helices α3, α4, α5 and loop α3-α4 of BAX, and when such an agent is determined to bind to the site, identifying the agent as a selective inhibitor of a BAX.

In an embodiment, the BAX binding site comprises one or more of residues Q76, 180, A81, V83, D84, T85, D86, P88, V91, F92, V95, F116, L119, L120 and K123. In an embodiment, the BAX binding site comprises all of residues Q76, 180, A81, V83, D84, T85, D86, P88, V91, F92, V95, F116, L119, L120 and K123. In an embodiment, the BAX binding site also comprises one or more of residues R34, D71, S72, M74, Q77, R78, 180, A81, A82, V83, D84, T85, D86, S87, P88, R89, V91, F92, V95, Y115, F116, S118, K119, L120, L122, K123, A124, C126, T127, K128, V129, L132, T135, 1136, and W139.

A method is also provided of identifying an agent as a selective inhibitor of a BCL-2, BCL-XL, MCL-1, BCL-B, BCL-w, pro-apoptotic BAK or BFL-1/A1 comprising

a) contacting the BCL-2, BCL-XL, MCL-1, BCL-B, BCL-w, or BFL-1/A1 with the agent and determining if the agent inhibits BCL-2, BCL-XL, MCL-1, BCL-B, BCL-w, or BFL-1/A1 activity, and
b) identifying the site of binding on a BCL-2, BCL-XL, MCL-1, BCL-B, BCL-w, or BFL-1/A1 of an agent identified in step a) as inhibiting BCL-2, BCL-XL, MCL-1, BCL-B, BCL-w, or BFL-1/A1, and determining if the site of binding substantially overlaps, or is co-extensive with, a binding site structurally equivalent to a binding site on a BAX comprised by helices α3, α4, α5 and loop α3-α4 of BAX,
wherein an agent that both (i) inhibits BCL-2, BCL-XL, MCL-1, BCL-B, BCL-w, or BFL-1/A1 and (ii) binds to a site structurally equivalent to a binding site on a BAX comprised by helices α3, α4, α5 and loop α3-α4 of BAX, is identified as a selective inhibitor of BCL-2, BCL-XL, MCL-1, BCL-B, BCL-w, or BFL-1/A1, respectively.

BCL-2, BCL-XL, MCL-1, BCL-B, BCL-w, or BFL-1/A1 are known to share sequence homology with BAX (see (9) for example). While BCL-2, BCL-XL, MCL-1, BCL-B, BCL-w, or BFL-1/A1 have different number of residues from BAX because of the differences in length of the loop between helix 1 and 2, the inhibitory site identified herein is not in this locations. Moreover, structures show conservation between polar, charge or hydrophobic residues and, more importantly, the surface topology of these proteins is similar to the inhibitory site defined for BAX herein.

As used herein, “BAX” is Bcl-2-associated x-protein. In an embodiment, the BAX is mammalian. In a preferred embodiment, the BAX is a human BAX. In an embodiment, the BAX comprises consecutive amino acid residues having the following sequence:

(SEQ ID NO: 1)
MDGSGEQPRGGGPTSSEQIMKTGALLLQGFIQDRAGRMGGEAPELALDPV
PQDASTKKLSECLKRIGDELDSNMELQRMIAAVDTDSPREVFFRVAADMF
SDGNFNWGRVVALFYFASKLVLKALCTKVPELIRTIMGWTLDFLRERLLG
WIQDQGGWDGLLSYFGTPTWQTVTIFVAGVLTASLTIWKKMG.

As used herein, “tBID” is truncated BH3 interacting-domain death agonist. For example, as generated by generated by Caspase-8 cleavage of BID. In an embodiment, the tBID is a human tBID.

In an embodiment of the methods described herein, the BAX is monomer. In an embodiment of the methods described herein, the BAX is inactive. In an embodiment of the methods described herein, the BAX is an oligomer.

In an embodiment of the methods described herein, the agent does not bind to helix α1, α6, α2 and/or α9 of BAX.

In an embodiment of the methods described herein, the methods are useful for identifying therapeutic cell death inhibitors.

In an embodiment of the methods described herein, the agent is a small molecule of 2000 daltons or less. In an embodiment of the methods described herein, the agent is a small molecule of 1500 daltons or less. In an embodiment of the methods described herein, the agent is a small molecule of 1000 daltons or less. In an embodiment of the methods described herein, the agent is a small molecule of 800 daltons or less. In an embodiment of the methods described herein, the agent is a small molecule of either 2000, 1500, 1000, 800, 700, 600, 500 or 400 daltons or less. In an embodiment of the methods described herein, the agent is a small organic molecule.

In an embodiment, “determining” as used herein means experimentally determining.

Also provided is a method of inhibiting Bcl-2-associated x-protein (BAX) comprising contacting the BAX with a compound having a structure listed in Table 1, or a pharmaceutically acceptable salt thereof or stereoisomer thereof, in an amount effective to inhibit BAX. In an embodiment, the BAX is in a subject and the compound is administered to the subject.

Also provided is a method of treating hypoxic cardiomyocytes or cardiac ischemia-reperfusion injury in a subject comprising administering to the subject an amount of a compound having a structure listed in Table 1, or an amount of iMAC or an amount of iMAC2, or a pharmaceutically acceptable salt of any thereof or stereoisomer thereof, in an amount effective to treat hypoxic cardiomyocytes or cardiac ischemia-reperfusion injury, respectively.

Also provided is a method of treating a condition associated with excessive BAX in a subject comprising administering to the subject an amount of a compound having a structure listed in Table 1, or an amount of iMAC1 or an amount of iMAC2, or a pharmaceutically acceptable salt of any thereof or stereoisomer thereof, in an amount effective to treat a condition associated with excessive BAX. In an embodiment, the condition associated with excessive BAX is selected from the group consisting of a cardiovascular diseases, a neurodegenerative disease, an immunological disorder, ischemia, infertility, a hematological disorders, renal hypoxia, hepatitis, asthma and AIDS.

In an embodiment, the subject is human.

Diseases associated with premature or unwanted cell death and are characterized with abnormal activation, or expression or function of BAX include: cardiovascular diseases and disorders (e.g. arteriosclerosis, heart failure, heart transplantation, aneurism, chronic pulmonary disease, ischemic heart disease, hypertension, thrombosis, cardiomyopathies), neurodegenerative diseases and neurological disorders (e.g. Alzheimer's disease, Parkinson's disease, Huntington's disease, retinitis pigmentosa, spinal muscular atrophy, various forms of cerebellar degeneration, amyotrophic lateral sclerosis), immunological disorders (e.g. organ transplant rejection, arthritis, lupus, IBD, Crohn's disease, asthma, multiple sclerosis, diabetes), ischemia (e.g. stroke, myocardial infarction and reperfusion injury), infertility (e.g. premature menopause, ovarian failure or follicular atresia), blood disorders (e.g. fanconi anemia, aplastic anemia, thalassemia, congenital neutropenia, myelodysplasia), renal hypoxia, hepatitis, asthma and AIDS. BAX inhibitors that target BAX through the site identified herein, including compounds listed in Table 1 below can be used for treatment or prevention of the aforementioned diseases.

Also provided is a method of identifying a compound as an inhibitor of a Bcl-2-associated x-protein (BAX) comprising

a) in silico modelling a pharmacophore for a binding site on BAX comprised by helices α³, α4, α5 and loop α3-α4 of BAX,
b) conducting an in silico comparison of the 3D structure of the compound with the modeled pharmacophore;
c) identifying the compound as an inhibitor or not of BAX,
wherein a compound having a 3D structure that matches or substantially overlaps the modeled pharmacophore is identified as an inhibitor of BAX, and a compound having a 3D structure that does not match or does not overlap the modeled pharmacophore is not identified as an inhibitor of BAX.

In an embodiment, steps of the methods are performed on a computer. A computer specifically programmed to perform the method is also an embodiment of the invention, as is a computer comprising a non-transitory program for performing the method. In an embodiment, the method further comprising testing a compound identified as an inhibitor of BAX by the in silico method in an animal model of a cardiovascular disease, a neurodegenerative disease, an immunological disorder, ischemia, infertility, a hematological disorders, renal hypoxia, hepatitis, asthma or AIDS, so as to determine the toxicity of the compound.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

Herein is disclosed an approach using NMR studies, molecular modeling, biophysical and biochemical assays, which has permitted characterization of two small molecule inhibitors of pro-apoptotic BAX, which effectively inhibit BAX-mediated pore formation. Remarkably, the small molecule inhibitors interact with monomeric BAX using a previously unidentified binding cleft on the surface of inactive BAX, which is distinct from those responsible for BAX activation.

The dysregulation of apoptosis is a key feature of a number of diseases including cancer, neurodegeneration, HIV and cardiovascular disease. BAX is a pro-apoptotic BCL-2 family protein that functions as a critical gateway to mitochondrial apoptosis and was discovered based on its heterodimeric interaction with anti-apoptotic BCL-2 (1). Despite their striking structural similarities, BAX and BCL-2 have opposing functions. Whereas BCL-2 is a resident mitochondrial outer membrane protein that blocks BAX through protein interaction, BAX is a cytosolic protein that, when triggered by cellular stress signals, undergoes a structural transformation and translocates to the mitochondria to form a putative homooligomeric pore, irreversibly damaging the mitochondria (2,3). Oligomerization of BAX and its close homologue, BAK, within the mitochondrial outer membrane enables the release of apoptogenic factors such as cytochrome c and smac/diablo that turn on caspases, the enzymatic effectors of apoptosis.

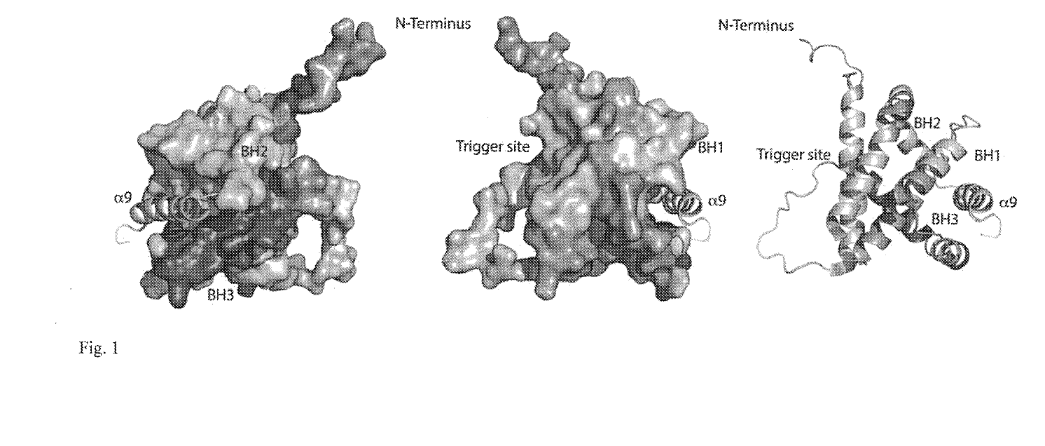

There are a number of structural features of BAX known to be vital to its regulation. The BAX trigger site (blue) (FIG. 1) is a recently discovered binding site for BH3-only proteins such as BIM and tBID, which results in activation of BAX, followed by its translocation to the outer mitochondrial membrane and release of apoptogenic factors. In contrast, the canonical BH3-binding pocket, identified in anti-apoptotic proteins, formed by BH domains 1-3 (green), maps to the opposite side of BAX, but remains occupied by the Cterminal helix 9 (yellow) when the protein is in the inactive, monomeric state (4). Finally the BH3 domain (dark green) of BAX is known to form an inhibitory complex with anti-apoptotic Bcl-2 family proteins, this interaction site is buried in inactive monomeric BAX and is only exposed after activation.

The discovery of the BAX trigger site has opened the door for the development of novel therapeutics which utilize this regulatory site to trigger cell death (5). While this would be beneficial in the treatment of a number of diseases where apoptosis is specifically repressed, a number of diseases involve unwanted apoptosis and would benefit from the ability to specifically inhibit BAX and block BAX-mediated apoptosis.

A screen for small molecule inhibitors of BAX-mediated cytochrome c release by Bombrun et al., identified the inhibitor of mitochondrial apoptotic channel (iMAC) family of molecules (6,7,8). The three molecules selected here impair BAX-mediated cell death (6,7) (FIG. 2).

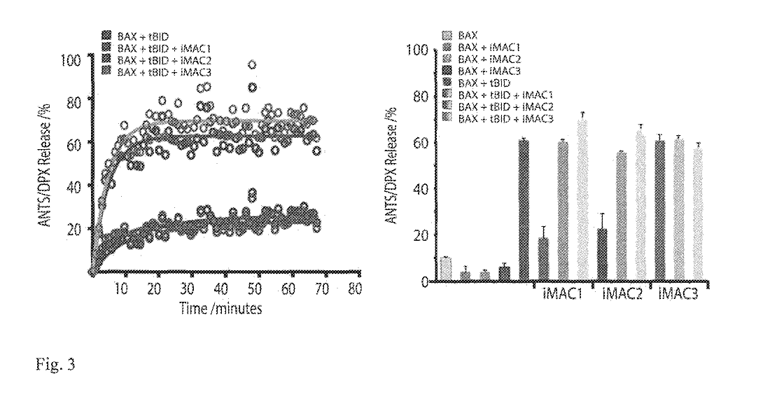

iMAC1 and iMAC2 Inhibit BAX Activation in Liposome Release Assays: Liposomes containing the fluorophore, ANTS, and the quencher, DPX, were incubated with 200 nM BAX activated with 30 nM tBID resulting in BAX-mediated pore formation in the liposomal membrane and release of ANTS and DPX. Both iMACS1 and iMAC2, but not iMAC3, inhibit BAX-mediated pore formation in liposomes, as shown in FIG. 3.

iMACs Bind to Both Monomeric and Oligomeric BAX in vitro. Ligand-observed NMR of the aromatic peaks of iMAC2 show chemical shift changes, protein to ligand STD transfer, and WATER LOGSY transfers demonstrating binding to both oligomeric BAX (FIG. 23(A)) and monomeric BAX (FIG. 23(B)).

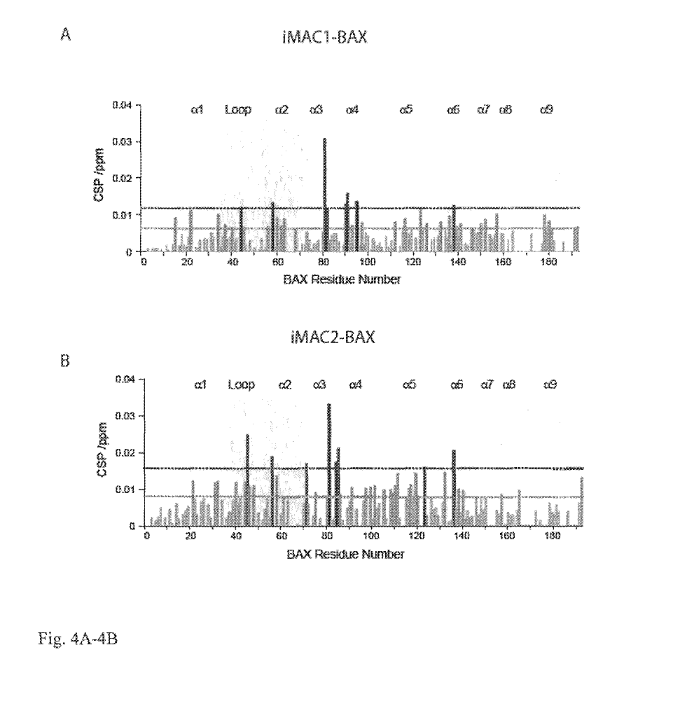

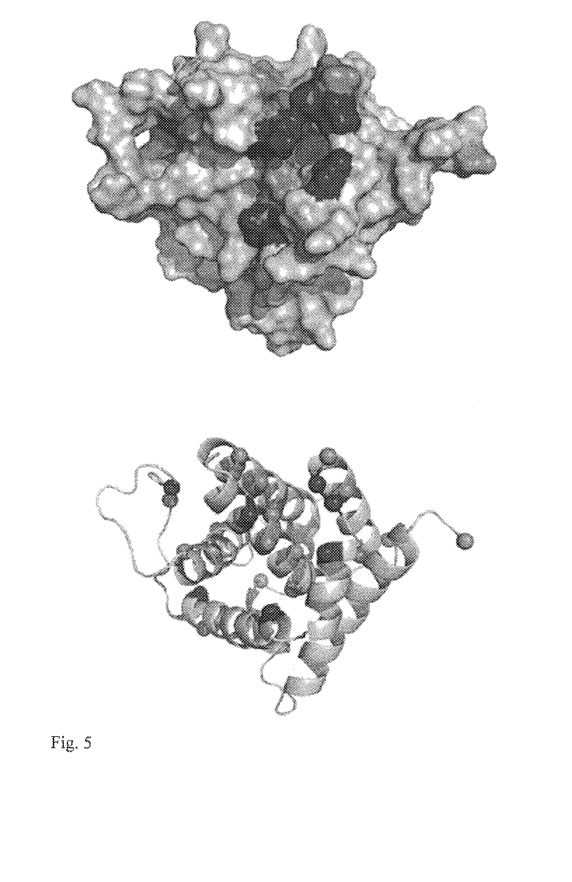

The iMAC Binding Site on Monomeric BAX Does Not Map to Any Known Binding-Site: FIG. 4 shows measured chemical shift perturbations (CSP) of ¹⁵N-BAX upon iMAC1 (4A) and iMAC2 (4B) titration up to a ratio of 3:1 BAX:iMACs are plotted as a function of BAX residue number. Significant CSP mapped onto the surface and ribbon depictions of BAX (FIG. 5), the BAX trigger site (blue), the canonical BH3-binding site and a9 (green) are included for comparison. Affected residues are represented by the orange and red spheres in the ribbon diagram and by the orange and red bars in the plot (the calculated significance threshold is marked in orange and is equal to the average shift plus the standard deviation while values at double the threshold are marked in red).

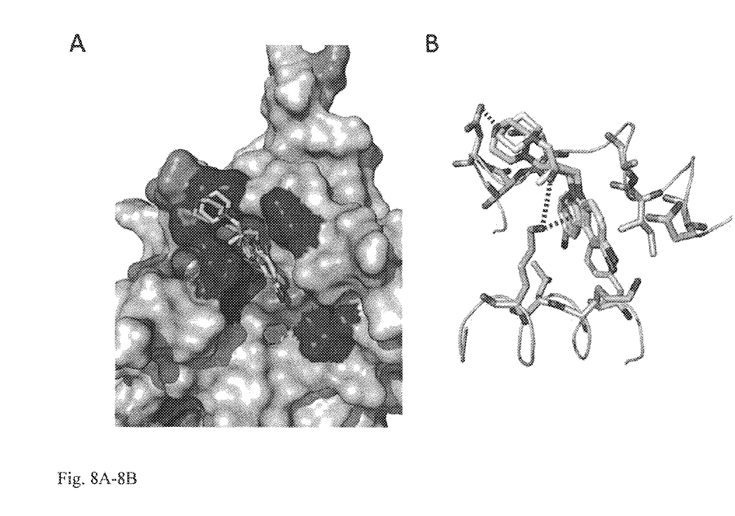

NMR and Molecular Docking Propose a Novel Inhibitory Binding Site. FIG. 8 shows NMR CSP—guided in silico docking of iMAC1 (green) and iMAC2 (red) with the BAX monomer NMR structure (PDB ID: 1F16) using Glide (Maestro, Shrodinger). (A) The lowest energy docking pose is consistent with the observed NMR CSP data (orange and red). (B) Key proposed interaction with the iMAC molecules, a number of hydrophobic contacts, cation-pi and electro static interactions contribute to the proposed binding site.

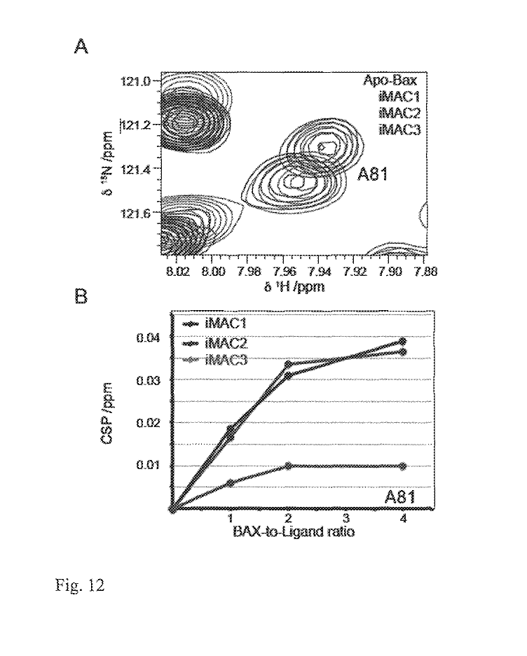

Only iMAC1 and iMAC2 Bind to the Novel BAX Inhibitory Site: In FIG. 12 Key binding site residues show chemical shift changes only for iMAC1 (green) and iMAC2 (red), but not for the liposome-inactive iMAC3 (purple), supporting the role of this site in the observed inhibition of BAX.

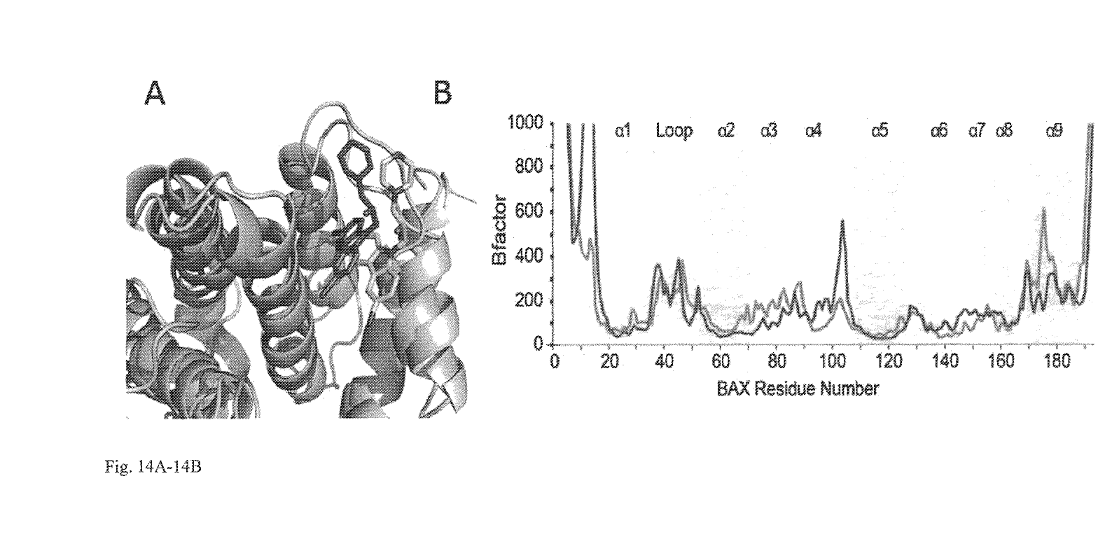

Molecular Dynamics Simulations Suggest iMACs Form Stable Interactions With the BAX Inhibitory Site: 20 ns molecular dynamics (MD) simulations were performed on the BAX-iMAC1 complex using Desmond (Maestro, Schrodinger) at 300 K and 350 K. After 20 ns of MD simulation, iMAC1 remains bound to the proposed binding pocket in contact with the same residues (See FIG. 14, panel A). Comparison of the MD derived B-factors from 20 ns simulations of BAX with and without iMAC1 show evidence for reduced motions in several important regions of secondary structure, including the BH3 domain and helix 9, in the presence of iMAC1 (See FIG. 14, panel B).

Using a combination of biochemical and NMR-based methods, a pair of small molecules have been identified that interact with inactive-monomeric BAX and inhibit the ability of BAX to form membrane pores in liposomes. The small molecules, known as iMACs, bind to a novel, inhibitory binding site on inactive BAX, which is unrelated to the previously identified regulatory interaction surfaces of BAX. The identification of this inhibitory binding site allows for identification of agents that inhibit unwanted apoptosis.

The following molecules have been identified based the in silico screen as small molecule BAX inhibitors that bind to the novel BAX site. An in silico library was generated from commercial vendors and NIH libraries using ZINC12 database (zinc.docking.org) to perform an in silico screen and identify small molecules which recognize the newly identified inhibitory site of BAX. Using the software Glide (Schrodinger) a library of 462,326 small molecules were screened for binding to the novel inhibitory BAX site using a multi-step protocol involving an initial low-precision, high-throughput docking protocol to select 50,000 candidates for standard precision docking with the best 10,000 candidates for submitted to extra-precision docking. A total of 6132 potential molecules were identified, these molecules were filtered for a suitable molecular weight (250-500 Daltons) and suitable hydrogen bonding properties (no more than 5 hydrogen bond donors and no more than 10 hydrogen bond acceptors) resulting in 1872 molecules. A partial list of molecules selected from the screen is shown in Table 1 below:

TABLE 1
Compounds selected by Screen

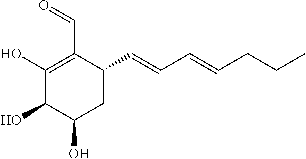 [SMILES: CCC/C=C/C=C/[C@@H]1C[C@@H](O)[C@@H](O)C(O)=C1C=O]

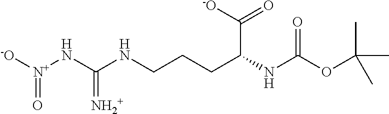

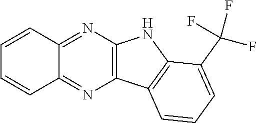 [SMILES: FC(F)(F)c1cccc2c1[nH]c1nc3ccccc3nc12]

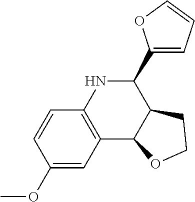 [SMILES: COc1ccc2c(c1)[C@@H]1OCC[C@@H]1[C@H](c1ccco1)N2]

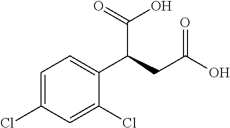 [SMILES: O=C(O)C[C@H](C(=O)O)c1ccc(Cl)cc1Cl]

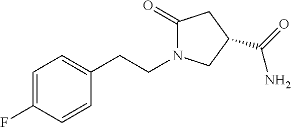 [SMILES: NC(=O)[C@H]1CC(=O)N(CCc2ccc(F)cc2)C1]

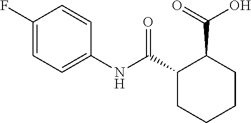 [SMILES: O=C(O)[C@H]1CCCC[C@@H]1C(=O)Nc1ccc(F)cc1]

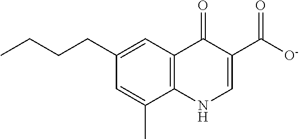 [SMILES: CCCCc1cc(C)c2[nH]cc(C(=O)[O-])c(=O)c2c1]

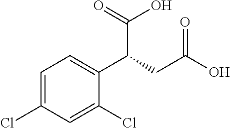 [SMILES: O=C(O)C[C@@H](C(=O)O)c1ccc(Cl)cc1Cl]

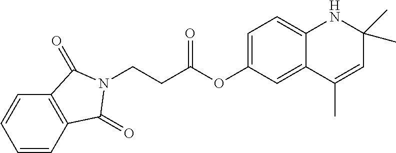 [SMILES: CC1=CC(C)(C)Nc2ccc(OC(=O)CCN3C(=O)c4ccccc4C3=O)cc21]

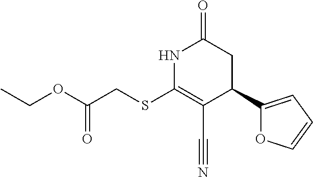 [SMILES: CCOC(=O)CSC1=C(C#N)[C@H](c2ccco2)CC(=O)N1]

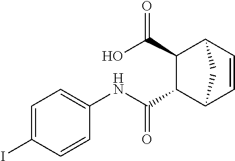 [SMILES: O=C(Nc1ccc(I)cc1)[C@@H]1[C@@H](C(=O)O)[C@H]2C=C[C@@H]1C2]

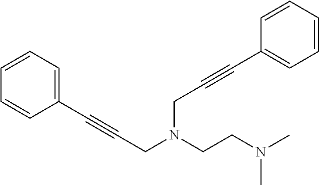 [SMILES: CN(C)CCN(CC#Cc1ccccc1)CC#Cc1ccccc1]

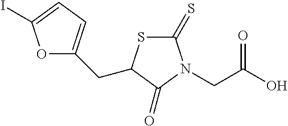 [SMILES: O=C(O)CN1C(=O)C(Cc2ccc(I)o2)SC1=S]

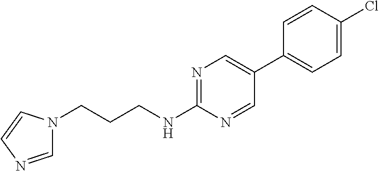 [SMILES: Clc1ccc(-c2cnc(NCCCn3ccnc3)nc2)cc1]

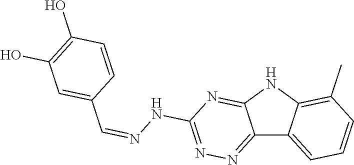 [SMILES: Cc1cccc2c1[nH]c1nc(N/N=C\c3ccc(O)c(O)c3)nnc12]

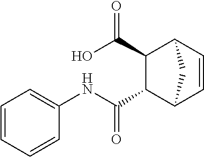 [SMILES: O=C(Nc1ccccc1)[C@@H]1[C@@H](C(=O)O)[C@H]2C=C[C@@H]1C2]

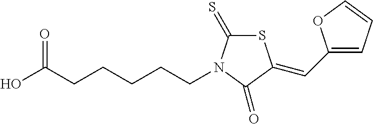 [SMILES: O=C(O)CCCCCN1C(=O)/C(=C/c2ccco2)SC1=S]

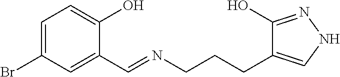 [SMILES: Oc1ccc(Br)cc1/C=N/CCCc1c[nH]nc1O]

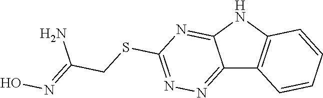 [SMILES: N/C(CSc1nnc2c(n1)[nH]c1ccccc12)=N\O]

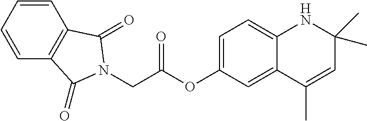 [SMILES: CC1=CC(C)(C)Nc2ccc(OC(=O)CN3C(=O)c4ccccc4C3=O)cc21]

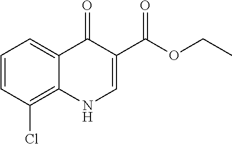 [SMILES: CCOC(=O)c1c[nH]c2c(Cl)cccc2c1=O]

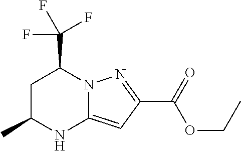 [SMILES: CCOC(=O)c1cc2n(n1)[C@H](C(F)(F)F)C[C@H](C)N2]

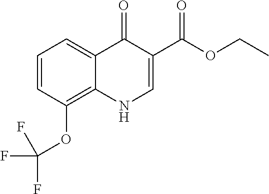 [SMILES: CCOC(=O)c1c[nH]c2c(OC(F)(F)F)cccc2c1=O]

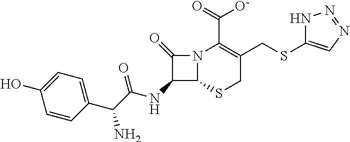 [SMILES: N[C@@H](C(=O)N[C@H]1C(=O)N2C(C(=O)[O-])=C(CSc3cnn[nH]3)CS[C@H]12)c1ccc(O)cc1]

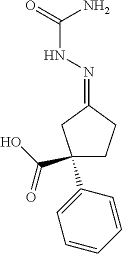 [SMILES: NC(=O)N/N=C1/CC[C@](C(=O)O)(c2ccccc2)C1]

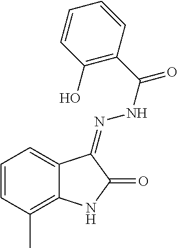 [SMILES: Cc1cccc2c1NC(=O)/C2=N\NC(=O)c1ccccc1O]

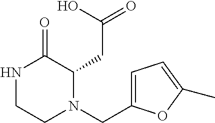 [SMILES: Cc1ccc(CN2CCNC(=O)[C@@H]2CC(=O)O)o1]

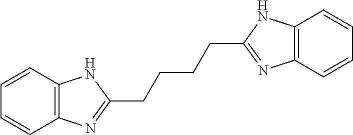 [SMILES: c1ccc2[nH]c(CCCCc3nc4ccccc4[nH]3)nc2c1]

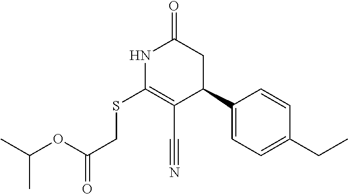 [SMILES: CCc1ccc([C@@H]2CC(=O)NC(SCC(=O)OC(C)C)=C2C#N)cc1]

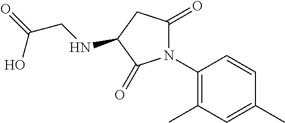 [SMILES: Cc1ccc(N2C(=O)C[C@H](NCC(=O)O)C2=O)c(C)c1]

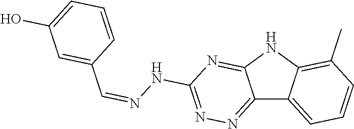 [SMILES: Cc1cccc2c1[nH]c1nc(N/N=C\c3cccc(O)c3)nnc12]

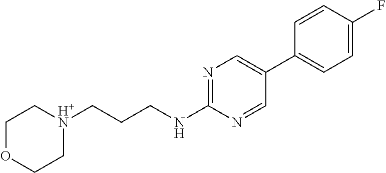

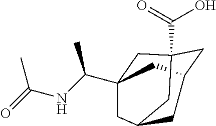 [SMILES: CC(=O)N[C@@H](C)[C@@]12C[C@@H]3C[C@@H](C[C@@](C(=O)O)(C3)C1)C2]

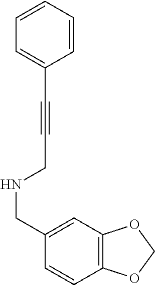 [SMILES: C(#Cc1ccccc1)CNCc1ccc2c(c1)OCO2]

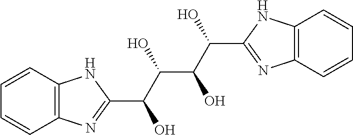 [SMILES: O[C@H]([C@H](O)[C@H](O)c1nc2ccccc2[nH]1)[C@@H](O)c1nc2ccccc2[nH]1]

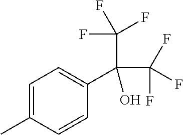 [SMILES: Cc1ccc(C(O)(C(F)(F)F)C(F)(F)F)cc1]

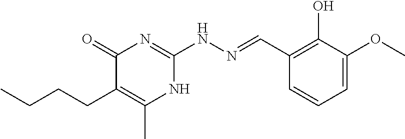 [SMILES: CCCCc1c(C)[nH]c(N/N=C/c2cccc(OC)c2O)nc1=O]

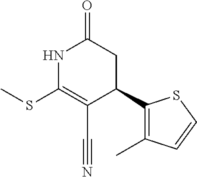 [SMILES: CSC1=C(C#N)[C@H](c2sccc2C)CC(=O)N1]

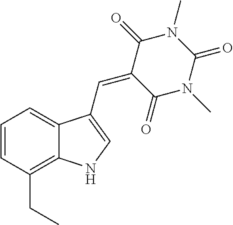 [SMILES: CCc1cccc2c(C=C3C(=O)N(C)C(=O)N(C)C3=O)c[nH]c12]

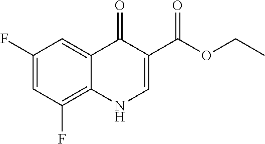 [SMILES: CCOC(=O)c1c[nH]c2c(F)cc(F)cc2c1=O]

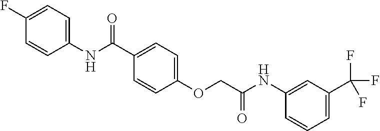 [SMILES: O=C(COc1ccc(C(=O)Nc2ccc(F)cc2)cc1)Nc1cccc(C(F)(F)F)c1]

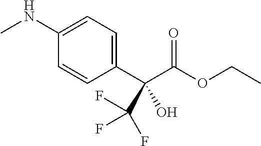 [SMILES: CCOC(=O)[C@](O)(c1ccc(NC)cc1)C(F)(F)F]

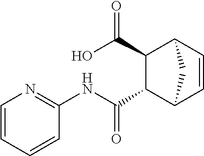 [SMILES: O=C(Nc1ccccn1)[C@@H]1[C@@H](C(=O)O)[C@H]2C=C[C@@H]1C2]

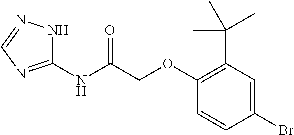 [SMILES: CC(C)(C)c1cc(Br)ccc1OCC(=O)Nc1ncn[nH]1]

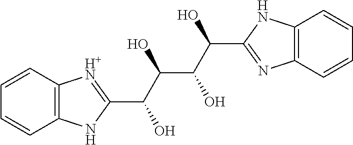

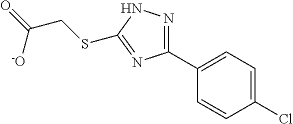

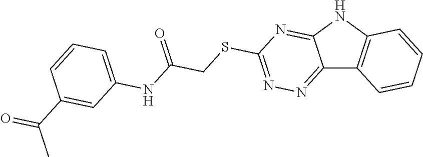 [SMILES: CC(=O)c1cccc(NC(=O)CSc2nnc3c(n2)[nH]c2ccccc23)c1]

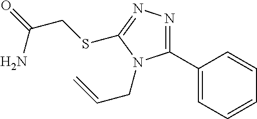 [SMILES: C=CCn1c(SCC(N)=O)nnc1-c1ccccc1]

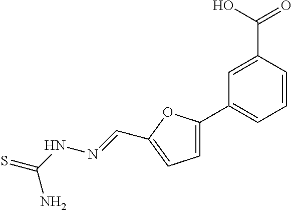 [SMILES: NC(=S)N/N=C/c1ccc(-c2cccc(C(=O)O)c2)o1]

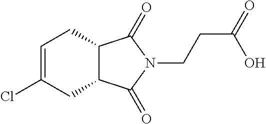 [SMILES: O=C(O)CCN1C(=O)[C@H]2CC=C(Cl)C[C@H]2C1=O]

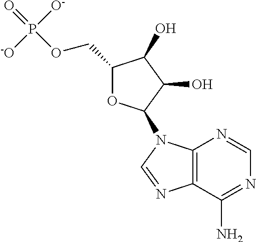

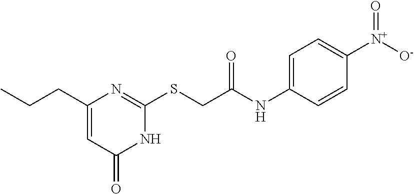 [SMILES: CCCc1cc(=O)[nH]c(SCC(=O)Nc2ccc([N+](=O)[O-])cc2)n1]

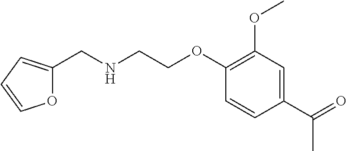 [SMILES: COc1cc(C(C)=O)ccc1OCCNCc1ccco1]

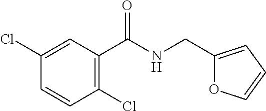 [SMILES: O=C(NCc1ccco1)c1cc(Cl)ccc1Cl]

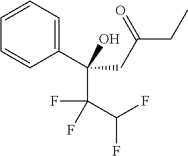 [SMILES: CCC(=O)C[C@@](O)(c1ccccc1)C(F)(F)C(F)F]

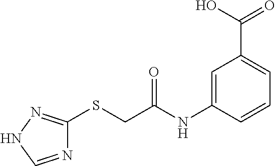 [SMILES: O=C(CSc1nc[nH]n1)Nc1cccc(C(=O)O)c1]

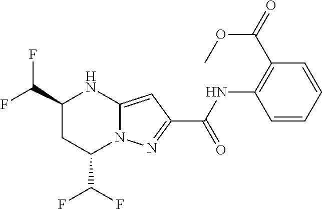 [SMILES: COC(=O)c1ccccc1NC(=O)c1cc2n(n1)[C@H](C(F)F)C[C@@H](C(F)F)N2]

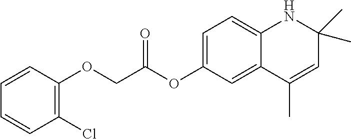 [SMILES: CC1=CC(C)(C)Nc2ccc(OC(=O)COc3ccccc3Cl)cc21]

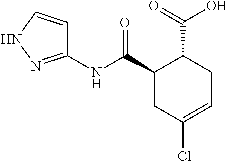 [SMILES: O=C(O)[C@@H]1CC=C(Cl)C[C@H]1C(=O)Nc1cc[nH]n1]

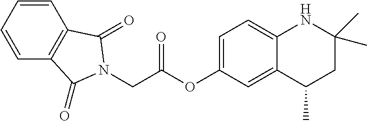 [SMILES: C[C@H]1CC(C)(C)Nc2ccc(OC(=O)CN3C(=O)c4ccccc4C3=O)cc21]

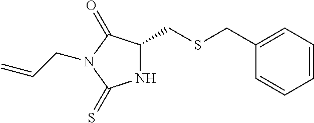 [SMILES: C=CCN1C(=O)[C@H](CSCc2ccccc2)NC1=S]

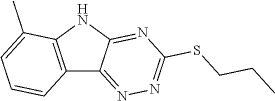 [SMILES: CCCSc1nnc2c(n1)[nH]c1c(C)cccc12]

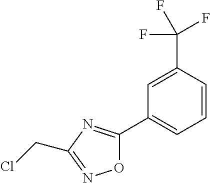 [SMILES: FC(F)(F)c1cccc(-c2nc(CCl)no2)c1]

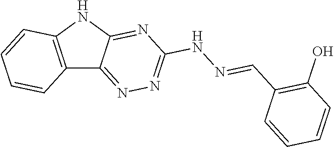 [SMILES: Oc1ccccc1/C=N/Nc1nnc2c(n1)[nH]c1ccccc12]

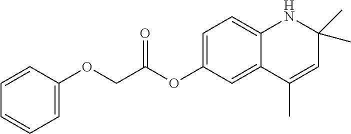 [SMILES: CC1=CC(C)(C)Nc2ccc(OC(=O)COc3ccccc3)cc21]

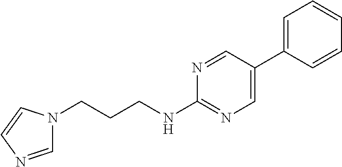 [SMILES: c1ccc(-c2cnc(NCCCn3ccnc3)nc2)cc1]

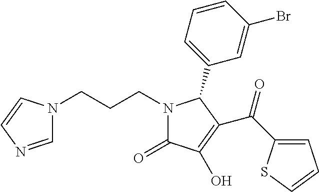 [SMILES: O=C(C1=C(O)C(=O)N(CCCn2ccnc2)[C@@H]1c1cccc(Br)c1)c1cccs1]

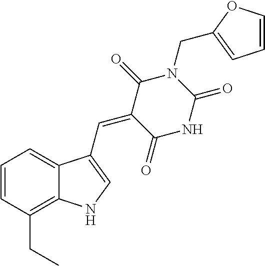 [SMILES: CCc1cccc2c(/C=C3\C(=O)NC(=O)N(Cc4ccco4)C3=O)c[nH]c12]

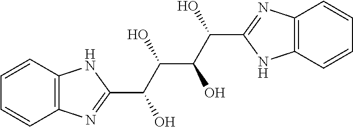 [SMILES: O[C@H]([C@H](O)[C@H](O)c1nc2ccccc2[nH]1)[C@H](O)c1nc2ccccc2[nH]1]

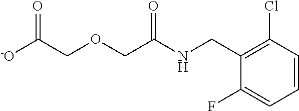

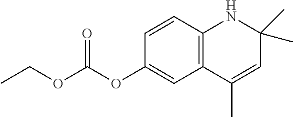 [SMILES: CCOC(=O)Oc1ccc2c(c1)C(C)=CC(C)(C)N2]

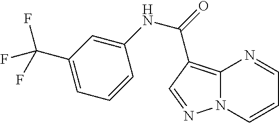 [SMILES: O=C(Nc1cccc(C(F)(F)F)c1)c1cnn2cccnc12]

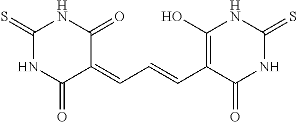 [SMILES: O=C1NC(=S)NC(=O)C1=C/C=C/c1c(O)[nH]c(=S)[nH]c1=O]

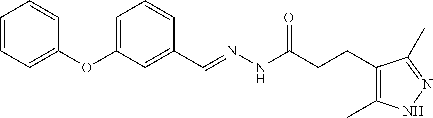 [SMILES: Cc1n[nH]c(C)c1CCC(=O)N/N=C/c1cccc(Oc2ccccc2)c1]

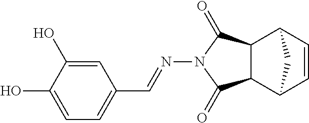 [SMILES: O=C1[C@@H]2[C@H](C(=O)N1/N=C/c1ccc(O)c(O)c1)[C@@H]1C=C[C@H]2C1]

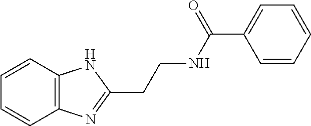 [SMILES: O=C(NCCc1nc2ccccc2[nH]1)c1ccccc1]

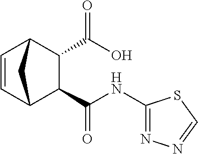 [SMILES: O=C(Nc1nncs1)[C@@H]1[C@@H](C(=O)O)[C@H]2C=C[C@@H]1C2]

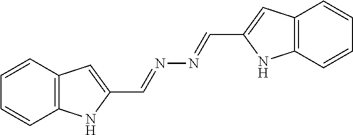 [SMILES: C(=N\N=C\c1cc2ccccc2[nH]1)/c1cc2ccccc2[nH]1]

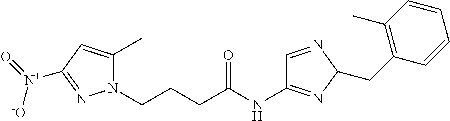

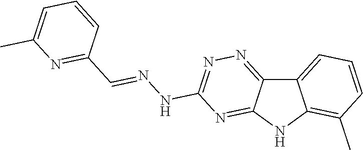 [SMILES: Cc1cccc(/C=N/Nc2nnc3c(n2)[nH]c2c(C)cccc23)n1]

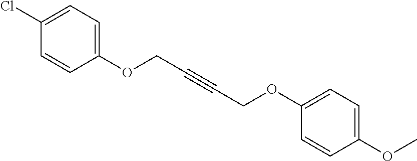 [SMILES: COc1ccc(OCC#CCOc2ccc(Cl)cc2)cc1]

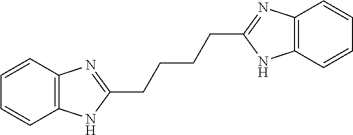 [SMILES: c1ccc2[nH]c(CCCCc3nc4ccccc4[nH]3)nc2c1]

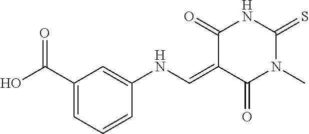 [SMILES: CN1C(=O)/C(=C/Nc2cccc(C(=O)O)c2)C(=O)NC1=S]

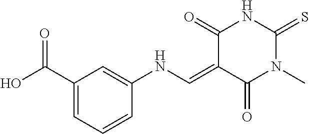 [SMILES: CN1C(=O)/C(=C/Nc2cccc(C(=O)O)c2)C(=O)NC1=S]

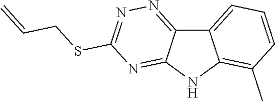 [SMILES: C=CCSc1nnc2c(n1)[nH]c1c(C)cccc12]

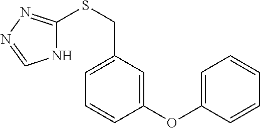 [SMILES: c1ccc(Oc2cccc(CSc3nnc[nH]3)c2)cc1]

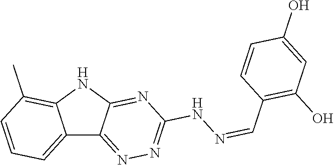 [SMILES: Cc1cccc2c1[nH]c1nc(N/N=C\c3ccc(O)cc3O)nnc12]

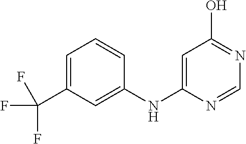 [SMILES: Oc1cc(Nc2cccc(C(F)(F)F)c2)ncn1]

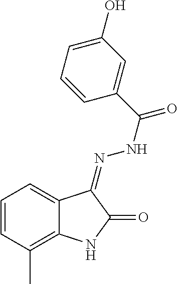 [SMILES: Cc1cccc2c1NC(=O)/C2=N\NC(=O)c1cccc(O)c1]

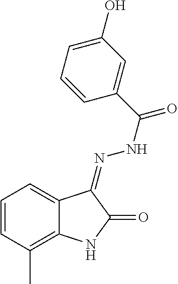 [SMILES: Cc1cccc2c1NC(=O)/C2=N\NC(=O)c1cccc(O)c1]

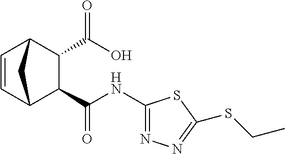 [SMILES: CCSc1nnc(NC(=O)[C@@H]2[C@@H](C(=O)O)[C@H]3C=C[C@@H]2C3)s1]

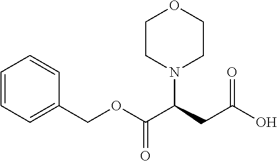 [SMILES: O=C(O)C[C@@H](C(=O)OCc1ccccc1)N1CCOCC1]

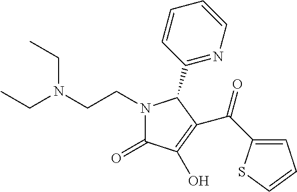 [SMILES: CCN(CC)CCN1C(=O)C(O)=C(C(=O)c2cccs2)[C@H]1c1ccccn1]

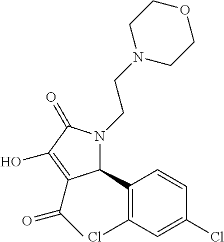 [SMILES: CC(=O)C1=C(O)C(=O)N(CCN2CCOCC2)[C@H]1c1ccc(Cl)cc1Cl]

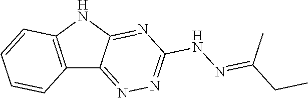 [SMILES: CC/C(C)=N/Nc1nnc2c(n1)[nH]c1ccccc12]

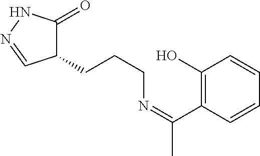 [SMILES: C/C(=N/CCC[C@@H]1C=NNC1=O)c1ccccc1O]

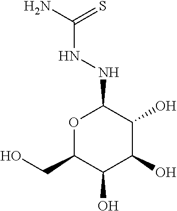 [SMILES: NC(=S)NN[C@@H]1O[C@H](CO)[C@H](O)[C@H](O)[C@H]1O]

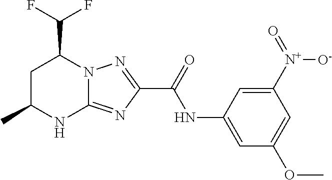

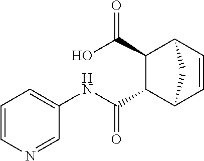 [SMILES: O=C(Nc1cccnc1)[C@@H]1[C@@H](C(=O)O)[C@H]2C=C[C@@H]1C2]

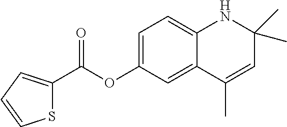 [SMILES: CC1=CC(C)(C)Nc2ccc(OC(=O)c3cccs3)cc21]

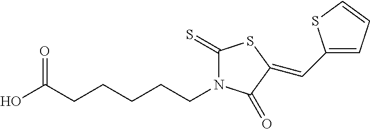 [SMILES: O=C(O)CCCCCN1C(=O)/C(=C/c2cccs2)SC1=S]

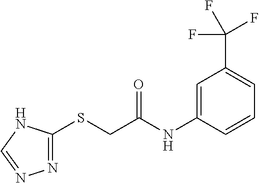 [SMILES: O=C(CSc1nnc[nH]1)Nc1cccc(C(F)(F)F)c1]

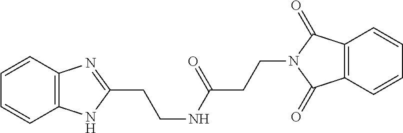 [SMILES: O=C(CCN1C(=O)c2ccccc2C1=O)NCCc1nc2ccccc2[nH]1]

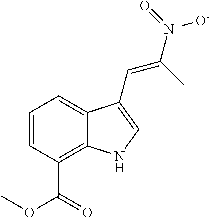 [SMILES: COC(=O)c1cccc2c(/C=C(\C)[N+](=O)[O-])c[nH]c12]

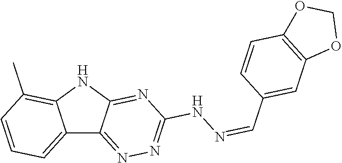 [SMILES: Cc1cccc2c1[nH]c1nc(N/N=C\c3ccc4c(c3)OCO4)nnc12]

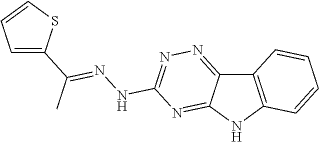 [SMILES: C/C(=N\Nc1nnc2c(n1)[nH]c1ccccc12)c1cccs1]

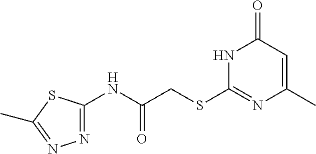

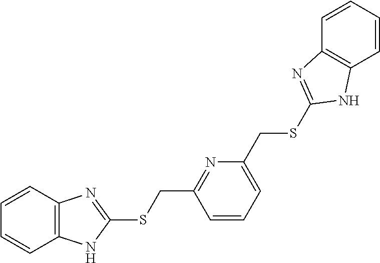 [SMILES: c1cc(CSc2nc3ccccc3[nH]2)nc(CSc2nc3ccccc3[nH]2)c1]

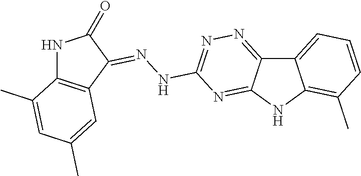 [SMILES: Cc1cc(C)c2c(c1)/C(=N\Nc1nnc3c(n1)[nH]c1c(C)cccc13)C(=O)N2]

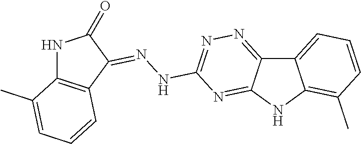 [SMILES: Cc1cccc2c1NC(=O)/C2=N/Nc1nnc2c(n1)[nH]c1c(C)cccc12]

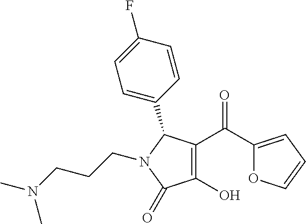 [SMILES: CN(C)CCCN1C(=O)C(O)=C(C(=O)c2ccco2)[C@H]1c1ccc(F)cc1]

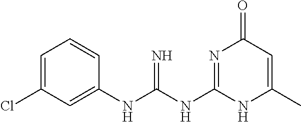 [SMILES: Cc1cc(=O)nc(NC(=N)Nc2cccc(Cl)c2)[nH]1]

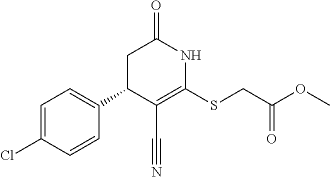 [SMILES: COC(=O)CSC1=C(C#N)[C@H](c2ccc(Cl)cc2)CC(=O)N1]

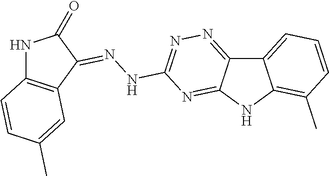 [SMILES: Cc1ccc2c(c1)/C(=N\Nc1nnc3c(n1)[nH]c1c(C)cccc13)C(=O)N2]

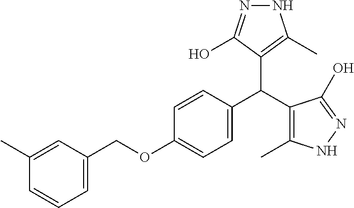 [SMILES: Cc1cccc(COc2ccc(C(c3c(O)n[nH]c3C)c3c(O)n[nH]c3C)cc2)c1]

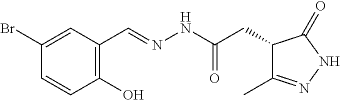 [SMILES: CC1=NNC(=O)[C@H]1CC(=O)N/N=C/c1cc(Br)ccc1O]

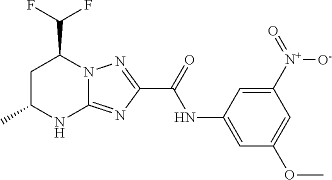 [SMILES: COc1cc(NC(=O)c2nc3n(n2)[C@H](C(F)F)C[C@@H](C)N3)cc([N+](=O)[O-])c1]

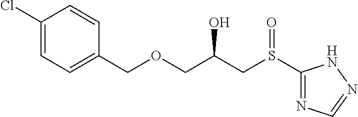 [SMILES: O=S(C[C@H](O)COCc1ccc(Cl)cc1)c1ncn[nH]1]

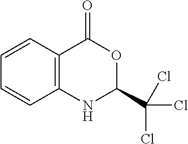 [SMILES: O=C1O[C@@H](C(Cl)(Cl)Cl)Nc2ccccc21]

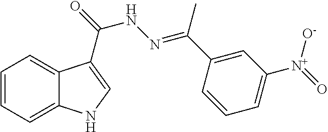 [SMILES: C/C(=N\NC(=O)c1c[nH]c2ccccc12)c1cccc([N+](=O)[O-])c1]

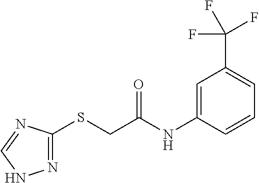 [SMILES: O=C(CSc1nc[nH]n1)Nc1cccc(C(F)(F)F)c1]

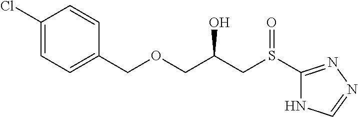 [SMILES: O=S(C[C@H](O)COCc1ccc(Cl)cc1)c1nnc[nH]1]

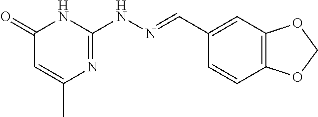 [SMILES: Cc1cc(=O)[nH]c(N/N=C/c2ccc3c(c2)OCO3)n1]

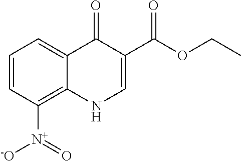

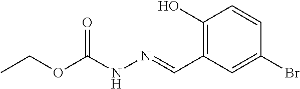 [SMILES: CCOC(=O)N/N=C/c1cc(Br)ccc1O]

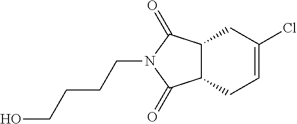 [SMILES: O=C1[C@H]2CC=C(Cl)C[C@H]2C(=O)N1CCCCO]

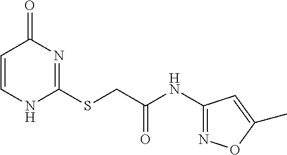 [SMILES: Cc1cc(NC(=O)CSc2nc(=O)cc[nH]2)no1]

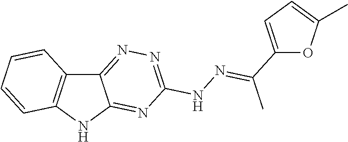 [SMILES: C/C(=N\Nc1nnc2c(n1)[nH]c1ccccc12)c1ccc(C)o1]

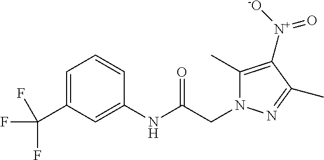

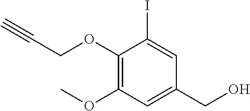

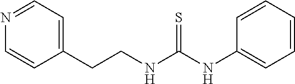 [SMILES: S=C(NCCc1ccncc1)Nc1ccccc1]

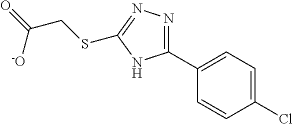

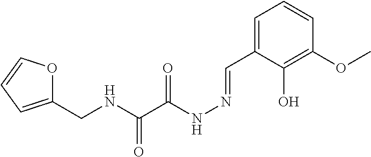 [SMILES: COc1cccc(/C=N/NC(=O)C(=O)NCc2ccco2)c1O]

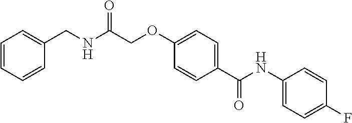 [SMILES: O=C(COc1ccc(C(=O)Nc2ccc(F)cc2)cc1)NCc1ccccc1]

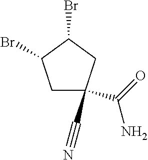 [SMILES: N#C[C@]1(C(N)=O)C[C@@H](Br)[C@@H](Br)C1]

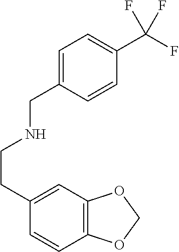 [SMILES: FC(F)(F)c1ccc(CNCCc2ccc3c(c2)OCO3)cc1]

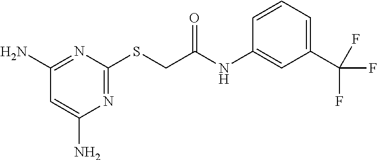 [SMILES: Nc1cc(N)nc(SCC(=O)Nc2cccc(C(F)(F)F)c2)n1]

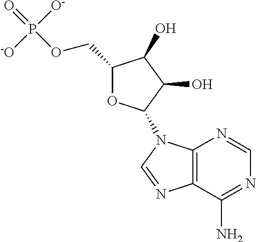

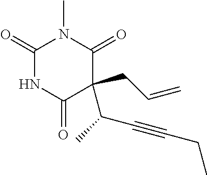 [SMILES: C=CC[C@@]1([C@@H](C)C#CCC)C(=O)NC(=O)N(C)C1=O]

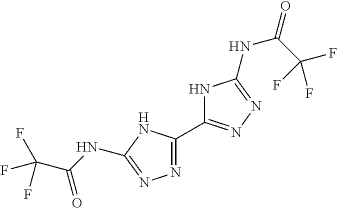 [SMILES: O=C(Nc1nnc(-c2nnc(NC(=O)C(F)(F)F)[nH]2)[nH]1)C(F)(F)F]

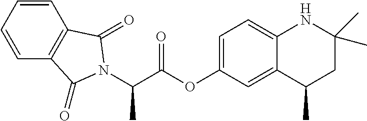 [SMILES: C[C@@H]1CC(C)(C)Nc2ccc(OC(=O)[C@@H](C)N3C(=O)c4ccccc4C3=O)cc21]

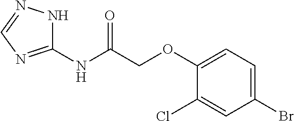 [SMILES: O=C(COc1ccc(Br)cc1Cl)Nc1ncn[nH]1]

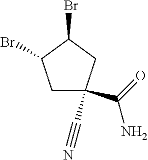

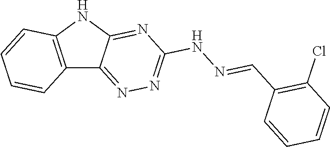 [SMILES: Clc1ccccc1/C=N/Nc1nnc2c(n1)[nH]c1ccccc12]

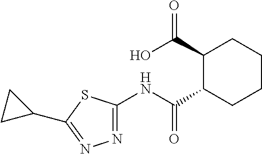 [SMILES: O=C(O)[C@H]1CCCC[C@@H]1C(=O)Nc1nnc(C2CC2)s1]

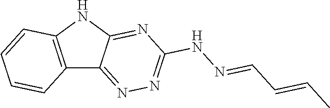 [SMILES: C/C=C/C=N/Nc1nnc2c(n1)[nH]c1ccccc12]

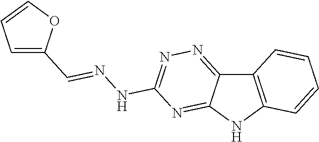 [SMILES: C(=N\Nc1nnc2c(n1)[nH]c1ccccc12)/c1ccco1]

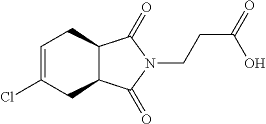 [SMILES: O=C(O)CCN1C(=O)[C@H]2CC(Cl)=CC[C@H]2C1=O]

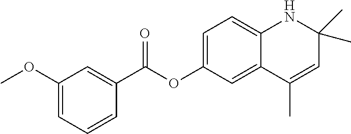

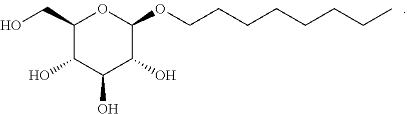 [SMILES: CCCCCCCCO[C@@H]1O[C@H](CO)[C@@H](O)[C@H](O)[C@H]1O]

Diseases associated with premature or unwanted cell death and are characterized with abnormal activation, or expression or function of BAX include: cardiovascular diseases and disorders (e.g. arteriosclerosis, heart failure, heart transplantation, aneurism, chronic pulmonary disease, ischemic heart disease, hypertension, thrombosis, cardiomyopathies), neurodegenerative diseases and neurological disorders (e.g. Alzheimer's disease, Parkinson's disease, Huntington's disease, retinitis pigmentosa, spinal muscular atrophy, various forms of cerebellar degeneration, amyotrophic lateral sclerosis), immunological disorders (e.g. organ transplant rejection, arthritis, lupus, IBD, Crohn's disease, asthma, multiple sclerosis, diabetes), ischemia (e.g. stroke, myocardial infarction and reperfusion injury), infertility (e.g. premature menopause, ovarian failure or follicular atresia), blood disorders (e.g. fanconi anemia, aplastic anemia, thalassemia, congenital neutropenia, myelodysplasia), renal hypoxia, hepatitis, asthma and AIDS. BAX inhibitors that target BAX through the site identified herein, including compounds listed in Table 1 from the in silico screen, can be used for treatment or prevention of the aforementioned diseases.

### Functional Activity

iMAC1 protects cardiac myocytes from cell death induced by hypoxia as compared to vehicle (0.2% DMSO), whereas iMAC1 has no effect on cardiac myocytes under normoxia conditions. Accordingly, it can be seen that inhibitor compounds which bind to the novel BAX site can protect cardiac myocytes from cell death induced by hypoxia.

### REFERENCES

1. Z. N. Oltvai, C. L. Milliman, S. J. Korsmeyer, Cell 74, 609 (1993).
2. M. C. Wei et al., Science 292, 727 (2001).
3. E. Gavathiotis et al., Mol. Cell. 40, 481 (2010).
4. E. Gavathiotis et al., Nature 455, 1076 (2008).
5. E. Gavathiotis et al., Nature Chem. Bio. 8, 639 (2012).
6. Peixoto et al., Biochem. J. 423, 381 (2009).
7. Peixoto et al., Mitochondrion 12, 14 (2012).
8. A. Bombrun et al, J. Med. Chem. 46, 4365 (2003).
9. Walensky et al., Trends in Biochemical Sciences, December 2011, Vol. 36, No. 12.

1. A method of identifying an agent as an inhibitor of a Bcl-2-associated x-protein (BAX) comprising
a) contacting the BAX with the agent and determining if the agent inhibits truncated BH3 interacting-domain death agonist (tBID)-induced BAX activation and/or BAX-mediated pore formation, and
b) identifying, for an agent identified in step a) as inhibiting tBID-induced BAX activation and/or BAX-mediated pore formation, the site on the BAX to which the agent binds, and determining if said site substantially overlaps, or is co-extensive with, a binding site comprised by helices α3, α4, α5 and loop α3-α4 of BAX,
wherein an agent that both (i) inhibits tBID-induced BAX activation and/or BAX-mediated pore formation and (ii) binds to a site that substantially overlaps, or is co-extensive with, a binding site comprised by helices α3, α4, α5 and loop α3-α4 of BAX, is identified as an inhibitor of BAX.

2. The method of claim 1, wherein the BAX binding site comprised by helices α3, α4, α5 and loop α3-α4 of BAX comprises one or more of residues Q76, 180, A81, V83, D84, T85, D86, P88, V91, F92, V95, F116, L119, L120 and K123.

3. The method of claim 1, wherein the BAX binding site comprises all of residues Q76, 180, A81, V83, D84, T85, D86, P88, V91, F92, V95, F116, L119, L120 and K123.

4. The method of claim 1, wherein the BAX binding site comprised by helices α3, α4, α5 and loop α3-α4 of BAX also comprises one or more of residues R34, D71, S72, M74, Q77, R78, 180, A81, A82, V83, D84, T85, D86, S87, P88, R89, V91, F92, V95, Y115, F116, S118, K119, L120, L122, K123, A124, C126, T127, K128, V129, L132, T135, 1136, and W139.

5. The method of claim 1, wherein the BAX is monomeric BAX.

6. The method of claim 1, wherein the BAX is a human BAX.

7. The method of claim 1, wherein the BAX is an alpha isoform BAX.

8. The method of claim 1, wherein inhibition of tBID-induced BAX activation and/or BAX pore formation is determined by a liposomal release assay.

9. The method of claim 1, wherein inhibition of tBID-induced BAX activation and/or BAX-mediated pore formation is determined by quantifying the reduction in release of fluorescent molecules encapsulated in liposomal membranes comprising BAX.

10. The method of claim 1, wherein identifying the site of binding on the BAX comprises determining chemical shift changes of ¹⁵N-BAX upon a detergent titration, below the detergent's critical micellular concentration (CMC) point, at one or more residues of the BAX.

11. The method of claim 10, wherein the detergent comprises octyl glucoside.

12-18. (canceled)

19. A method of inhibiting Bcl-2-associated x-protein (BAX) comprising contacting the BAX with a compound having a structure listed in Table 1, or a pharmaceutically acceptable salt thereof or stereoisomer thereof, in an amount effective to inhibit BAX.

20. The method of claim 19, wherein the BAX is in a subject and the compound is administered to the subject.

21. A method of treating hypoxic cardiomyocytes or cardiac ischemia-reperfusion injury in a subject comprising administering to the subject an amount of a compound having a structure listed in Table 1, or an amount of iMAC1 or an amount of iMAC2, or a pharmaceutically acceptable salt of any thereof or stereoisomer thereof, in an amount effective to treat hypoxic cardiomyocytes or cardiac ischemia-reperfusion injury, respectively.

22-23. (canceled)

24. The method of claim 21, wherein the subject is human.

25. The method of claim 21, wherein iMAC1 or iMAC2 are administered.

26-27. (canceled)
